SEQUENCE LISTING (14 sequences)

SEQ ID NO: 1 - protein, 1106 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 2 - protein, 20 aa, synthetic construct
METDTLLLWVLLLWVPGSTG

SEQ ID NO: 3 - protein, 511 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 4 - protein, 9 aa, synthetic construct
YPYDVPDYA

SEQ ID NO: 5 - protein, 8 aa, synthetic construct
DYKDDDDK

SEQ ID NO: 6 - protein, 10 aa, synthetic construct
EQKLISEEDL

SEQ ID NO: 7 - protein, 5 aa, synthetic construct
GSGGS

SEQ ID NO: 8 - protein, 4 aa, synthetic construct
GGGS

SEQ ID NO: 9 - protein, 4 aa, synthetic construct
GGSG

SEQ ID NO: 10 - protein, 5 aa, synthetic construct
GGSGG

SEQ ID NO: 11 - protein, 5 aa, synthetic construct
GSGSG

SEQ ID NO: 12 - protein, 5 aa, synthetic construct
GSGGG

SEQ ID NO: 13 - protein, 5 aa, synthetic construct
GGGSG

SEQ ID NO: 14 - protein, 5 aa, synthetic construct
GSSSG

(19) United States
(12) Patent Application Publication
(10) Pub. No.: US 2024/0408137 A1
(43) Pub. Date: Dec. 12, 2024

(54) METHODS FOR MAKING AND USING THERAPEUTIC CELLS

(71) Applicant: THE REGENTS OF THE UNIVERSITY OF CALIFORNIA, Oakland, CA (US)

(72) Inventors: Yvonne Yu-Hsuan CHEN, Los Angeles, CA (US); Christopher M. WALTHERS, Los Angeles, CA (US); Beata BERENT-MAOZ, Los Angeles, CA (US); Mobina ROSHANSDELL, Los Angeles, CA (US); Brenda Ji, Los Angeles, CA (US)

(73) Assignee: THE REGENTS OF THE UNIVERSITY OF CALIFORNIA, Oakland, CA (US)

(21) Appl. No.: 18/696,543

(22) PCT Filed: Sep. 29, 2022
(86) PCT No.: PCT/US22/77283

§ 371 Date: Mar. 28, 2024

Related U.S. Application Data
(60) Provisional application No. 63/250,790, filed on Sep. 30, 2021.
(60) Provisional application No. 63/322,050, filed on Mar. 21, 2022.
(60) Provisional application No. 63/405,331, filed on Sep. 9, 2022.

(51) Int. Cl. A61K 35/17; A61K 39/00; A61P 35/02; C07K 14/725; C07K 16/28; C12N 5/0783

(52) U.S. Cl. CPC A61K 35/17; A61K 39/4611; A61K 39/4631; A61K 39/464412; A61K 39/464424; A61P 35/02; C07K 14/7051; C07K 16/2803; C07K 16/2887; C12N 5/0636; A61K 2239/13; A61K 2239/21; A61K 2239/28; C07K 2317/31; C07K 2317/622; C07K 2317/73; C07K 2319/03; C07K 2319/33; C12N 2501/2302; C12N 2501/2315; C12N 2510/00

(57) ABSTRACT

The current application fulfills a need for methods of making bi-specific CAR T-cells that have high efficacy. Aspects of the disclosure relate to a method for manufacturing a CD19/CD20 bi-specific chimeric antigen receptor (CAR) T cell comprising the ordered steps of: (a) providing a composition comprising a population of cells comprising T cells; (b) contacting the composition comprising the population of cells comprising T cells with one or more of a transactivating composition, IL-2, and/or IL-15; (c) transducing the cell composition from (b) with a CD19/CD20 bi-specific CAR nucleic acid; and (d) removing the transactivating composition from the cell composition of (c). Further aspects relate to a population of CD19/CD20 bi-specific CAR-T cells produced by the methods of the disclosure. Yet further aspects describe a method for treating a subject for B-cell lymphoma comprising administering cells of the disclosure.

This application claims priority of U.S. Provisional Patent Application No. 63/250,790, filed Sep. 30, 2021; U.S. Provisional Patent Application No. 63/322,050, filed Mar. 21, 2022; and U.S. Provisional Patent Application No. 63/405,331, filed Sep. 9, 2022, all of which are hereby incorporated by reference in their entirety.

### SEQUENCE LISTING

The application contains a Sequence Listing prepared in compliance with ST.26 format and is hereby incorporated by reference in its entirety. Said Sequence Listing, created on Sep. 23, 2022 is named UCLAP0134WO_Seq_Listing and is 14,520 bytes in size.

### BACKGROUND OF THE INVENTION

### I. Field of the Invention

This invention relates to the field of methods of cancer biology and cellular therapies.

### II. Background

Chimeric antigen receptors (CARs) are artificial molecules that redirect the specificity of T cells to predetermined antigens. These receptors are frequently used to graft the specificity of a monoclonal antibody onto a T cell; with transfer of their coding sequence facilitated by retroviral or lentiviral vectors. Using adoptive transfer, autologous T cells can be genetically modified ex vivo to express a CAR specific for a cancer cell of interest. The T cells, which can then recognize and kill the cancer cells, are reintroduced into the patient. Phase I clinical studies of this approach have shown efficacy.

The most common form of CARs are fusions of single-chain variable fragments (scFv) derived from monoclonal antibodies, fused to CD3-zeta signaling domain, which contains 3 ITAMs. CD3-zeta may not provide a fully competent activation signal and additional co-stimulatory signaling is needed. For example, chimeric CD28 and OX40 can be used with CD3-Zeta to transmit a proliferative/survival signal, or all three can be used together. Such molecules result in the transmission of a zeta signal in response to recognition by the scFv of its target.

Multiple clinical trials have reported remarkable therapeutic efficacy of anti-CD19 CAR-modified T cells against both acute and chronic B-cell malignancies. However, multiple cases have also been reported of patients relapsing with the emergence of CD19-negative leukemia or lymphoma (Maude et al. 2014). There is a need for the development of more effective CAR therapies that address antigen escape.

### SUMMARY

The current application fulfills a need by providing methods for making bi-specific CAR T-cells that are shown to have high efficacy. Aspects of the disclosure relate to a method for manufacturing a CD19/CD20 bi-specific chimeric antigen receptor (CAR) T cell comprising the ordered steps of two, three, four, or all of: (a) providing a composition comprising a population of cells comprising T cells; (b) enriching the cell population for CD14−/CD25−/CD62L+ cells; (c) contacting the composition comprising the enriched cell population with one or more of a transactivating composition, IL-2, and/or IL-15; (d) transducing the cell composition from (c) with a CD19/CD20 bi-specific CAR nucleic acid; and (c) removing the transactivating composition from the transduced cell composition of (d). Further aspects relate to a population of CAR-T cells produced by the methods of the disclosure and compositions comprising the population of cells. Yet further aspects describe a method for treating a subject for B-cell malignancy comprising administering cells of the disclosure. Further aspects relate to a method for manufacturing a CD19/CD20 bi-specific chimeric antigen receptor (CAR) T cell comprising the ordered steps of two, three, or all of: (a) providing a composition comprising a population of cells comprising T cells (b) contacting the composition comprising the population of cells with one or more of a transactivating composition, IL-2, and/or IL-15; (c) transducing the composition comprising the population of cells from (b) with a CD19/CD20 bi-specific CAR nucleic acid; (d) removing the transactivating composition from the transduced cell composition of (c); and wherein the transactivating composition comprises MACS® GMP T cell TransAct™. Yet further aspects relate to a method for manufacturing a CD19/CD20 bi-specific chimeric antigen receptor (CAR) T cell comprising the ordered steps of two, three, or all of: (a) providing a composition comprising a population of cells comprising T cells (b) contacting the composition comprising the population of cells with one or more of a transactivating composition, IL-2, and/or IL-15; (c) transducing the composition comprising the population of cells from (b) with a CD19/CD20 bi-specific CAR nucleic acid; (d) removing the transactivating composition from the transduced cell composition of (c); and wherein the method further comprises contacting the cell with protamine sulfate within 24 hours of the cell transduction.

Further aspects relate to a method for manufacturing a (CAR) T cell comprising the ordered steps of: (a) providing a composition comprising a population of cells comprising T cells; (b) contacting the composition comprising the population of cells with one or more of a transactivating composition, IL-2, and/or IL-15; (c) transducing the composition comprising the population of cells from (b) with a nucleic acid encoding a CAR; (d) removing the transactivating composition from the transduced cell composition of (c); and wherein the method further comprises contacting the cell with protamine sulfate within 24 hours of the cell transduction. Further aspects relate to a method for manufacturing a (CAR) T cell comprising the ordered steps of: (a) providing a composition comprising a population of cells comprising T cells; (b) contacting the composition comprising the population of cells with one or more of a transactivating composition, IL-2, and/or IL-15; (c) transducing the composition comprising the population of cells from (b) with a nucleic acid encoding a CAR; (d) removing the transactivating composition from the transduced cell composition of (c); and wherein the transactivating composition comprises MACS® GMP T cell TransAct™. In some aspects, the CAR T cell comprises a CD19/CD20 bi-specific CAR T cell and/or the nucleic acid encodes a CD19/CD20 bi-specific CAR.

In some aspects, the CAR T cell comprises a TGF-β mono-specific, TGF-β bispecific, TGF-β tri-specific, BCMA mono-specific, BCMA bi-specific, BCMA tri-specific, BCMA/CS1 bi-specific, IL13Ra2/TGF-β bi-specific, IL13Rα2 mono-specific, IL13Rα2 bi-specific, IL13Rα2 tri-specific, GD2 mono-specific, GD2 bi-specific, GD2 tri-specific, EGFRvIII mono-specific, EGFRvIII bi-specific, EGFRvIII tri-specific, IL13Rα2/GD2 bi-specific, IL13Rα2/EGFRvIII bi-specific, GD2/EGFRvIII bi-specific, GD2/TGF-β bi-specific, TYRP1 mono-specific, TYRP1 bi-specific, TYRP1 tri-specific, or a TYRP1/TGF-β bi-specific CAR. The IL13Rα2 CAR may comprise a IL13 polypeptide. The CAR may comprise an scFv that is specific for the cancer antigen. In some aspects, a CAR comprising an IL13Rα2 binding region comprises a IL13 polypeptide, such as an IL13 mutein.

Further aspects relate to a method for manufacturing a CD19/CD20 bi-specific chimeric antigen receptor (CAR) T cell comprising the ordered steps of two, three, or all of: (a) providing a composition comprising a population of cells comprising T cells (b) contacting the composition comprising the population of cells with one or more of a transactivating composition. IL-2, and IL-15; (c) transducing the composition comprising the population of cells from (b) with a CD19/CD20 bi-specific CAR nucleic acid; (d) removing the transactivating composition from the transduced cell composition of (c); wherein the transactivating composition comprises MACS® GMP T cell TransAct™; and wherein the concentration of IL-2 is 50 IU/mL and/or the concentration of IL-15 is 0.5 ng/ml. Yet further aspects relate to a method for manufacturing a CD19/CD20 bi-specific chimeric antigen receptor (CAR) T cell comprising the ordered steps of two, three, or all of: (a) providing a composition comprising a population of cells comprising T cells (b) contacting the composition comprising the population of cells with one or more of a transactivating composition, IL-2, and/or IL-15; (c) transducing the composition comprising the population of cells from (b) with a CD19/CD20 bi-specific CAR nucleic acid; (d) removing the transactivating composition from the transduced cell composition of (c); wherein the method further comprises contacting the cell with protamine sulfate within 24 hours of the cell transduction; and wherein the concentration of IL-2 is 50 IU/mL and/or the concentration of IL-15 is 0.5 ng/mL.

Further aspects relate to a method for manufacturing a CD19/CD20 bi-specific chimeric antigen receptor (CAR) T cell comprising the ordered steps of two, three, or all of: (a) providing a composition comprising a population of cells comprising T cells (b) contacting the composition comprising the population of cells with one or more of a transactivating composition, IL-2, and IL-15; (c) transducing the composition comprising the population of cells from (b) with a CD19/CD20 bi-specific CAR nucleic acid; (d) removing the transactivating composition from the transduced cell composition of (c); wherein the transactivating composition comprises MACS® GMP T cell TransAct™ diluted to 1:35; and wherein the concentration of IL-2 is 50 IU/mL and/or the concentration of IL-15 is 0.5 ng/mL. Yet further aspects relate to a method for manufacturing a CD19/CD20 bi-specific chimeric antigen receptor (CAR) T cell comprising the ordered steps of two, three, or all of: (a) providing a composition comprising a population of cells comprising T cells (b) contacting the composition comprising the population of cells with one or more of a transactivating composition, IL-2, and/or IL-15; (c) transducing the composition comprising the population of cells from (b) with a CD19/CD20 bi-specific CAR nucleic acid; (d) removing the transactivating composition from the transduced cell composition of (c); wherein the method further comprises contacting the cell with 10-20 μg/mL protamine sulfate within 24 hours of the cell transduction; wherein the concentration of IL-2 is 50 IU/mL and/or the concentration of IL-15 is 0.5 ng/mL.

The cells or population of cells of the disclosure may be maintained in a cell culture medium throughout the methods of the claims. At any step of the process, the cell culture medium may be serum-free. In some aspects, the process maintains the cells in serum-free medium throughout all of the steps. In some aspects, the methods of the claim exclude contacting the cells with serum. In some aspects, the step, as defined above, as step (a), (b), (c), (d), and/or (c) is a step in which the cells are in serum-free medium. The method may comprise or further comprise evaluating the population of cells comprising T cells for the CD14 and/or CD25 cell marker. The percentage of CD14+ and CD25+ cells may be determined or evaluated to be greater than or equal to 5%. The percentage of CD14+ and CD25+ cells evaluated or determined may be less than or equal to about 5%. The percentage of CD14+ and CD25+ cells may be determined or evaluated to be exactly, greater than, or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any derivable range therein. The method may comprise or further comprise depleting the population of cells of CD14+ and/or CD25+ cells; the depleted population of cells may be reduced by about, at least about, or at most about 60, 65, 70, 75, 80, 85, 90, 95, 96, 97, 98, 99 or 100% (or any range derivable therein) with respect to cells having one or both of CD14 and CD25. Depleting the population of cells of CD14+ and/or CD25+ cells may comprise or further comprise contacting the cells with anti-CD14 and/or anti-CD25 microbeads. In some aspects, the cells are not depleted for CD14+ or CD25+ cells.

The methods, such as the step of contacting a composition comprising the enriched cell population with one or more of a transactivating composition, IL-2, and/or IL-15; may comprise contacting the composition with a transactivating composition; in some aspects, the transactivating composition comprises MACS® GMP T cell TransAct™. MACS® GMP T cell TransAct™ is available commercially through, for example, Miltenyi Biotec. The product format is described as a polymeric nanomatrix conjugated to recombinant humanized CD3 and CD28 agonist supplied in phosphate buffered-saline (PBS), containing 0.03% poloxamer 188 and 5 g/L recombinant human serum albumin, pH 7.3-7.9. The capacity of the reagent is sufficient to activate and expand up to 2×10⁸ enriched T cells or up to 4×10⁸ PBMC in a maximal volume of 70 mL, when used at recommended titer of 1:17.5. The transactivating composition may be used at a titer of 1:25-1:40. In some aspects, the composition is diluted to 1:35. In some aspects the dilution or titer of the transactivating composition may be, be at least, or be at most 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or 1:50.

In some aspects, the population of cells comprising T cells comprises cells that have been isolated from a patient, such as a human patient, by leukapheresis. Leukapheresis is a laboratory procedure in which white blood cells are separated from a sample of blood. It is a specific type of apheresis, the more general term for separating out one particular constituent of blood and returning the remainder to the circulation. Leukapheresis may be performed to decrease a very high white blood cell count, to obtain blood cells from a patient (autologous) or donor (allogeneic) for later transplant into the patient, or to obtain cells for research purposes. Leukapheresis may be performed to obtain the patient's own blood cells for a later transplant. In aspects of the disclosure, the population of cells comprising T cells are human cells. In aspects of the disclosure, the population of cells comprising T cells are primary cells.

In some aspects, the step of transducing the cell composition with a nucleic acid encoding a CAR is performed 24-72 hours after the step of: contacting the cells with one or more of a transactivating composition, IL-2, and/or IL-15. In some aspects, the step of transducing the cell composition with a CAR nucleic acid is performed at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours, or any derivable range therein, after the step of contacting the cells with one or more of a transactivating composition, IL-2, and/or IL-15.

In some aspects, the IL-2 is in contact with the cells at a concentration of 50 IU/mL. In some aspects, the IL-2 is in contact with the cells at a concentration of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 IU/mL, or any derivable range therein. In some aspects, the cells are contacted with IL-15 and wherein the concentration of IL-15 in contact with the cells is 0.5 ng/ml. In some aspects, the IL-15 is in contact with the cells at a concentration of at least, at most, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 ng/ml, or any derivable range therein. In some aspects, the concentration of IL-2 is 50 IU/mL and the concentration of IL-15 is 0.5 ng/mL.

In some aspects, the step of removing the transactivating composition from the cell composition of transduced cells is performed 6-7 days after contacting the cells with one or more of a transactivating composition, IL-2, and/or IL-15. In some aspects, the step of removing the transactivating composition from the transduced cells is performed at least, at most, or exactly 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 17.25, 17.5, 17.75, 18, 18.25, 18.5, 18.75, 19, 19.25, 19.5, 19.75, 20, 20.25, 20.5, 20.75, or 21 days (or any derivable range therein) after contacting cells with one or more of a transactivating composition, IL-2, and/or IL-15. In some aspects, the methods comprise contacting the composition with the transactivating composition, IL-2, and IL-14. In aspects, of the disclosure, the “removal” of a compound or molecule happens when the cell media comprising the compound or molecule is replaced with media that is absent of the compound for a period of time that is significant, such as for at least 12 hours, 1 day, 2, days, or 3 days. On the contrary, the cells may be considered in continuous contact with a compound or molecule if the cell media comprising the compound or molecule is replaced with cell media that comprises the compound or molecule in a close time-frame, such as within 1 or 2 hours.

The cells may be transduced with a viral vector. The viral vector may be a lentiviral-based virus comprising a nucleic acid encoding the CAR. The virus may be packaged in a packaging cell. In some aspects, the virus is packaged in HEK-293T cells. The methods may comprise or further comprise contacting the cells with protamine sulfate. In some aspects, the protamine sulfate is contacted within 24 hours of cell transduction. In some aspects, the protamine sulfate is contacted within, before, or after exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96 hours or any derivable range therein of performing or within performing the cell transduction.

The protamine sulfate may be at a concentration of 10-20 μg/mL while in contact with the cells. In some aspects, the concentration of protamine sulfate is at a concentration of at least, of at most, or of exactly 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL, or any derivable range therein.

The cells may be transduced in a composition comprising protamine sulfate, IL-2, IL-15, and a virus comprising a nucleic acid encoding the CAR. The nucleic acid may comprise a lentivral vector backbone or a vector backbone that is based on the lentivirus. Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). The nucleic acid may comprise an epHIV7 vector backbone. Other suitable viral vectors include, for example, pRSV-Rev, pMDLg/pRRE, psPAX2, pCMV delta R8.2, pMD2.G, pCMV-VSV-G, pCMV-dR8.2 dvpr, pCI-VSVG, pCPRDEnv, pLTR-RD114A, pLenti-III (Applied Biological Materials; cat #LV587); 87 pLentiCRISPR v.1 (Addgene; cat #52963); 88 p156RRLsinppt (Addgene; cat #42795); 89 pFUGW (Addgene; cat #14883); 90 pFUG (Addgene; cat #14882); 90 pHAGE (Addgene; cat #46793); 91 pHRsin (Addgene; cat #12265); 92 pLenti (AMP) (Addgene; cat #61422); 93 pLK0.1 (Addgene; cat #10878); 94 pLL3.7 m (Addgene; cat #89362); 95 Puro.cre (Addgene; cat #17408); 96 pRSIEG (Cellecta; SVSHU6EG-L); pLenti7.3 (Thermo Fisher Scientific; cat #V53406); pLenti (OriGene; cat #PS100109); pSF_Lenti (Sigma; cat #OGS269); pLV-GFPSpark (Sinobiological; cat #LVCV-01); pLVX (Takara; cat #632164); pALD-Lenti (Aldevron), pLenti (Vigene; cat #P100020), pLenti CMV (Addgene), and pLV. In some aspects, the vector backbone does not contain an antibiotic resistance gene. In some aspects, the vector backbone does not contain a beta-lactam resistance gene.

In some aspects, the nucleic acid encodes for a CAR polypeptide having the sequence of SEQ ID NO:1 or a sequence with at least 80% sequence identity to SEQ ID NO:1. The nucleic acid may encode for a CAR having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:1. In some aspects, the nucleic acid encodes for a CAR polypeptide having an anti-CD19/CD20 binding region, and wherein the anti-CD19/CD20 binding region comprises SEQ ID NO:3 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:3. In some aspects, the nucleic acid encodes for a CAR polypeptide comprising an IgG4 hinge region, CD28 transmembrane region, 4-1BB costimulatory region, and a CD3-zeta intracellular signaling domain. In some aspects, the CAR polypeptide consists of an anti-CD19/CD20 binding region, IgG4 hinge region, CD28 transmembrane region, 4-1BB costimulatory region, and a CD3-zeta intracellular signaling domain. In some aspects, the CAR polypeptide consists of a signal peptide, an anti-CD19/CD20 binding region, IgG4 hinge region, CD28 transmembrane region, 4-1BB costimulatory region, and a CD3-zeta intracellular signaling domain.

The multiplicity of infection (MOI) may be 0.1-1.5. In some aspects, the MOI is at least, at most, or exactly 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2, 7, 2.8, 2.9, 3, 3, 1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10, or any derivable range therein. The cells may be transduced at a concentration of 1×10⁶ cells/mL. In some aspects, the transduced cells are at a concentration of at least, at most, or exactly 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or 1×10¹⁴ cells/mL (or any derivable range therein).

In some aspects, the cells are incubated with virus, protamine sulfate, IL-2 and/or IL-15 for 6-12 hours. In some aspects, the cells are incubated with virus, protamine sulfate, IL-2 and/or IL-15 for exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96 hours or any derivable range therein. The cells may be expanded after the transduction. The cells may be expanded 1.5-25 folds after transduction. In some aspects, the cells are expanded to, to at least, or to at most 2, 3, 4, 5, 6, 7, 8, 9, 10, 11, 12, 13, 14, 15, 16, 17, 18, 19, or 20 fold (or any derivable range therein) after transduction.

Methods of the disclosure may also exclude one or more enrichment steps. The methods may exclude an enrichment or depletion step after contacting cells with one or more of a transactivating composition, IL-2, and/or IL-15. The methods may comprise or further comprise cryopreserving the cells, The cells may be cryopreserved at a concentration of 1×10⁶ cells/mL-15×10⁶ cells/mL, In some aspects, the cells are cryopreserved at a concentration of, of at least, or of at most 1×10³, 2×10³, 3×10³, 4×10³, 5×10³, 6×10³, 7×10³, 8×10³, 9×10³, 1×10⁴, 2× 10⁴, 3×10⁴, 4×10⁴, 5×10⁴, 6×10⁴, 7×10⁴, 8×10⁴, 9×10⁴, 1×10⁵, 2×10⁵, 3×10⁵, 4× 10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9× 10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9× 10¹³, or 1×10¹⁴ cells/mL (or any derivable range therein). The cells may be cryopreserved at a time period of less than 17 days after transduction. In some aspects, the cells are cryopreserved at a time period of exactly, or of less than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days (or any derivable range therein) after transduction. The cells may be filtered prior to cryopreservation.

In some aspects, the cells are thawed cells. The cells, such as the populations of cells described herein may comprise at least 5% CD62L+CAR-T+ cells. In some aspects, the cells, such as the populations of cells described herein, comprises, comprises at least, comprises at most, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) CD62L+CAR-T+ cells. The transduced cells or transduced cell populations may have an average of 1-3 copies of the nucleic acid encoding the CAR per cell. In some aspects, the cells or cell populations have an average of, of at least, or of at most 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8 (or any derivable range therein) copies of the nucleic acid encoding the CAR per transduced cell.

In some aspects, the cells or cell populations comprise at least 70% viable cells after thawing. In some aspects, the cells or cell populations comprise or comprise at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% viable cells (or any derivable range therein) after thawing.

In some aspects, at least 10% of the cells in the cell populations are CD3+CAR+ cells. In some aspects, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 78, 79, 80, 81, 82, 83, 84, 85, 86, 87, 88, 89, 90, 91, 92, 93, 94, 95, 96, 97, 98, 99, or 100% (or any derivable range therein) of the cells in the cell populations are CD3+CAR+ cells. In some aspects, the population comprises a mixture of CD4+ and CD8+ T cells, such as CD4+ single positive and CD8+ single positive T cells. In some aspects, the ratio of CD8+ cells to CD4+ cells is about 3:1. In some aspects, the ratio of CD8+ cells to CD4+ cells may be, may be at least, or may be at most 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20 to 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20 (or any derivable range therein).

The population of cells may comprise at least 5% CD4+ cells. In some aspects, the population of cells may comprise, comprise at least, or comprise at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% CD4+ cells. The population of cells may comprise at least 15% CD8+ cells. In some aspects, the population of cells may comprise, comprise at least, or comprise at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% CD8+ cells. In some aspects, the ratio of CD4+ to CD8+ in the cells is not significantly changed from a control, wherein a control comprises the ratio of CD4+ to CD8+ in an untransduced sample from the patient.

In aspects of the disclosure, 1×10⁶-2×10⁸ cells are administered. In some aspects, the amount of cells administered to a subject may be, may be at least, or may be at most 1× 102, 2× 102, 3× 102, 4× 102, 5×10², 6×10², 7×10², 8× 102, 9× 102, 1×10³, 2×10³, 3× 10³, 4×10³, 5× 10³, 6× 10³, 7× 10³, 8×10³, 9× 10³, 1× 10⁴, 2× 10⁴, 3× 10⁴, 4×10⁴, 5× 10⁴, 6× 10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1× 10⁶, 2× 10⁶, 3× 10⁶, 4×10⁶, 5× 10⁶, 6× 10⁶, 7× 10⁶, 8×10⁶, 9×10⁶, 1×10⁷, 2× 10⁷, 3× 10⁷, 4× 10⁷, 5× 10⁷, 6× 10⁷, 7× 10⁷, 8×10⁷, 9× 10⁷, 1×10⁸, 2× 10⁸, 3×10⁸, 4×10⁸, 5×10⁸, 6× 10⁸, 7× 10⁸, 8× 10⁸, 9×10⁸, 1× 10⁹, 2× 10⁹, 3× 10⁹, 4×10⁹, 5× 10⁹, 6×10⁹, 7×10⁹, 8× 10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9× 10¹⁰, 1× 10¹¹, 2× 10¹¹, 3× 10¹¹, 4×10¹¹, 5×10¹¹, 6×10¹¹, 7× 10¹¹, 8×10¹¹, 9× 10¹¹, 1× 10¹², 2× 10¹², 3× 10¹², 4× 10¹², 5× 10¹², 6× 10¹², 7× 10¹², 8×10¹², 9×10¹², 1×10¹³, 2× 10¹³, 3×10¹³, 4× 10¹³, 5× 10¹³, 6×10¹³, 7×10¹³, 8×10¹³, 9× 10¹³, or 1×10¹⁴ cells (or any derivable range therein). The cells may be determined to be or evaluated as positive for expression of the CAR. In some aspects, the cells are autologous cells. In some aspects, the B-cell malignancy is relapsed/refractory B-cell malignancy. In some aspects, the subject has previously been treated for the B-cell malignancy. In some aspects, the subject has previously been treated with at least 1, at least 2, or at least 3 lines of therapy. In some aspects, the subject has previously been treated with or with at least 1, 2, 3, 4, 5, or 6 lines of therapy. In some aspects, the previous treatment comprises Bendamustine, Rituximab, Acalabrutinib, Umbralisib, Ublituximab, Lenalidomide, cyclophosphamide, doxorubicin, vincristine, prednisone, R-CHOP (combination of rituximab, cyclophosphamide, doxorubicin, vincristine, and prednisone), ifosfamide, carboplatin, etoposide, R-ICE (combination of rituximab, ifosfamide, carboplatin, and etoposide), R-EPOCH (combination of rituximab, etoposide phosphate, prednisone, vincristine sulfate, cyclophosphamide, and doxorubicin hydrochloride), ROR1-Targeting Antibody-Drug Conjugate, anti-CD19 bispecific T cell engager; gemcitabine, oxaliplatin, dexamethasone, autologous stem cell transplantation, or combinations thereof. The previous treatment may include the combination of bendamustine and rituximab. In some aspects, the previous treatment comprises the combination of umbralisib and ublituximab, In some aspects, the previous treatment comprises lenalidomide and rituximab. In some aspects, the previous treatment comprises rituximab, gemcitabine, and oxaliplatin. In some aspects, the previous treatment comprises rituximab and dexamethasone. In some aspects, the previous treatment comprises rituximab, cyclophosphamide, and etoposide. In some aspects, the previous treatment comprises the combination of rituximab, gemcitabine, dexamethasone, and carboplatin. In some aspects, the subject has not previously been treated for the B-cell malignancy. The therapy may be an additional therapy described herein, such as an immunotherapy, inhibition of co-stimulatory molecules, dendritic cell therapy, CAR-T cell therapy, adoptive T-cell therapy, chemotherapy, radiotherapy, or surgery.

The B-cell malignancy may be a lymphoma or leukemia. In some aspects, the B-cell malignancy comprises non-Hodgkin B-cell lymphoma. In some aspects, the non-Hodgkin B-cell lymphoma is further classified as indolent non-Hodgkin lymphomas, follicular lymphoma, lymphoplasmacytic lymphoma, marginal zone lymphoma, nodal marginal zone lymphoma, gastric mucosa-associated lymphoid tissue (MALT) lymphoma, extragastric MALT lymphoma, mediterranean abdominal lymphoma, splenic marginal zone lymphoma, primary cutaneous anaplastic large cell lymphoma, diffuse large B-cell lymphoma, primary mediastinal B-cell lymphoma, follicular large cell lymphoma, anaplastic large cell lymphoma, cutaneous anaplastic large cell lymphoma, systemic anaplastic large cell lymphoma, extranodal NK-/T-cell lymphoma, lymphomatoid granulomatosis, angioimmunoblastic T-cell lymphoma, peripheral T-cell lymphoma, hepatosplenic T-cell lymphoma, subcutaneous panniculitis-like T-cell lymphoma, enteropathy-type intestinal T-cell lymphoma, intravascular large B-cell lymphoma, Burkitt lymphoma, lymphoblastic lymphoma, adult T-cell leukemia/lymphoma, mantle cell lymphoma, posttransplantation lymphoproliferative disorder, true histiocytic lymphoma, primary effusion lymphoma, or plasmablastic lymphoma. In some aspects, the B-cell malignancy comprises leukemia, and wherein the leukemia is further classified as chronic lymphocytic leukemia, small-lymphocytic leukemia, acute lymphocytic leukemia, acute myeloid leukemia, chronic myeloid leukemia, chronic myelomonocytic leukemia, pediatric leukemia, precursor B acute lymphoblastic leukemia, precursor T acute lymphoblastic leukemia, Burkitt's leukemia, acute biphenotypic leukemia, B-cell prolymphocytic leukemia, acute promyelocytic leukemia, acute myeloblastic leukemia, acute megakaryoblastic leukemia, hairy cell leukemia, T-cell prolymphocytic leukemia, large granular lymphocytic leukemia, adult T-cell leukemia, or clonal eosinophilias.

In some aspects, the subject is being treated with an additional therapy. In some aspects, the method may further comprise administration of an additional therapy. The additional therapy may be an additional therapy described herein, such as an immunotherapy, inhibition of co-stimulatory molecules, dendritic cell therapy, CAR-T cell therapy, adoptive T-cell therapy, chemotherapy, radiotherapy, or surgery.

In some aspects, the additional therapy comprises a chemotherapy. In some aspects, the additional therapy comprises a lymphodepletion. The additional therapy may comprise fludarabine and/or cyclophosphamide. In some aspects, the chemotherapy comprises both fludarabine and cyclophosphamide. The additional therapy may be given prior to administration of the cells. In some aspects, the additional therapy is given after administration of the cells. The additional therapy may be given to the subject at a time period of five days prior to administration of the cells. In some aspects, the additional therapy is given to the subject at a time period of, of at least, or of at most 1, 2, 3, 4, 5, 6, 7, 8, 9, 10, 11, or 12 (or any derivable range therein) days prior to or after administration of the cells.

In some aspects, the subject is administered 30 mg/m²/day for 30 min of fludarabine for three days. In some aspects, the subject is administered 500 mg/m²/day for 60 min of cyclophosphamide for three days. In some aspects, the subject is administered, is administered at least, or is administered at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, (or any derivable range therein) mg/m²/day of fludarabine for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 minutes (or any derivable range therein) for a time period of, of at least, or of at most 1, 2, 3, 4, 5, 6, 7, 8, 9, or 10 days (or any derivable range therein). In some aspects, the subject is administered, is administered at least, or is administered at most 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000, (or any derivable range therein) mg/m²/day of cyclophosphamide for 10, 15, 20, 25, 30, 35, 40, 45, 50, 55, 60, 65, 70, 75, 80, 85, 90, 95, 100, 105, 110, 115, 120, 125, 130, 135, 140, 145, or 150 minutes (or any derivable range therein) for a time period of, of at least, or of at most 1, 2, 3, 4, 5, 6, 7, 8, 9, or 10 days (or any derivable range therein).

In some aspects, the method further comprises further purifying the CAR-expressing cells based on inclusion or exclusion of other cell markers, for example CD4, CD8, CD45RA, CD45RO, CCR7/CD197, CD62L, CD27, CD28, and CD1a or CD7, CD25, CD45, CD45RA, CD127, and CD200R.

The compositions and methods described herein may be modified so that the method is for preparing a T cell with a certain phenotype. In some aspects, the methods are for preparing a T cell with the phenotype: CD4⁺CD8⁻ T cells, CD4⁻CD8⁺ T cells, CD34⁺CD7⁺CD1a⁺ cells, CD3+ TCRab+, CD3+ TCRgd+, CD3+ TCRab+CD4+CD8−, CD3+ TCRab+CD8+CD4−, CD3+ TCRab+CD4+CD8−CD45RO-CD45RA+, CD3+ TCRab+CD8+CD4−CD45RO−CD45RA+, CD3+ TCRab+CD4+CD8−CD45RO−CD45RA+ CCR7+, CD3+ TCRab+CD8+CD4−CD45RO−CD45RA+ CCR7+, CD3+ TCRab+CD4+CD8−CD45RO−CD45RA+CD27+, CD3+ TCRab+CD8+CD4−CD45RO−CD45RA+CD27+, CD34⁺CD7⁺CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof.

In some aspects, the cells are further defined as having the following phenotype: CD4⁺CD8⁻ T cells, CD4⁻CD8⁺ T cells, CD34⁺CD7⁺CD1a⁺ cells, CD3+ TCRab+, CD3+ TCRgd+, CD3+ TCRab+CD4+CD8−, CD3+ TCRab+CD8+CD4−, CD3+ TCRab+CD4+CD8−CD45RO−CD45RA+, CD3+ TCRab+CD8+CD4−CD45RO−CD45RA+, CD3+ TCRab+CD4+CD8−CD45RO−CD45RA+ CCR7+, CD3+ TCRab+CD8+CD4−CD45RO−CD45RA+ CCR7+, CD3+ TCRab+CD4+CD8−CD45RO−CD45RA+CD27+, CD3+ TCRab+CD8+CD4−CD45RO−CD45RA+CD27+, CD34⁺CD7⁺CD1a⁺ cells, CD34+CD5+CD7+, CD34+CD5+CD7−, natural killer T cells, regulatory T cells, antigen-specific T cells, intraepithelial lymphocyte T cells, or cells that are CD45+, CD11b+, CD11b−, CD15+, CD15−, CD24+, CD24−, CD114+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD31+, CD31−, CD30+, CD30−, CD38+, and/or CD38− or combinations thereof.

The subject may be any animal, in particular a mouse, non-human primate, or human. In further aspects, the subject may have been determined to have or be at risk for cancer.

In some aspects, the cell population or composition of cells comprises a ratio of at least or at most 1:0.1, 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10, 1:10.5, 1:11, 1:11.5, 1:12, 1:12.5, 1:13, 1:13.5, 1:14, 1:14.5, 1:15, 1:20, 1:25, 1:30, 1:35, 1:40, 1:45, or 1:50 (or any range derivable therein) of live cells:cells having a phenotype and/or cell marker described herein or ratio of cells having a phenotype and/or cell marker described herein:live cells.

The compositions of the disclosure may comprise pharmaceutical excipients that are suitable for intravenous injection. In some aspects, the compositions comprise an injection solution, such as Isolyte®. Each 100 mL of Isolyte® S pH 7.4 (Multi-Electrolyte Injection) may comprise one or more of 0.53 g sodium chloride, 0.5 g sodium gluconate, 0.37 g sodium acetate trihydrate, 0.037 g potassium chloride, 0.03 g magnesium chloride hexahydrate, 0.012 g dibasic sodium phosphate heptahydrate, 0.00082 g monobasic potassium phosphate with a pH adjusted to 7.4. In some aspects, the composition comprises one or more of sodium chloride, sodium gluconate, sodium acetate, potassium chloride, magnesium chloride, dibasic sodium phosphate, and potassium phosphate. In some aspects, the composition is buffered at a pH of 7.0-7.8. In some aspects, the composition is at a pH of 7.4.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more.” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise.” “consist essentially of.” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: CD19/CD20 CAR T cell manufacturing schematic.

FIG. 2A-C. Design of phase-1 clinical trial evaluating CD19/CD20 bispecific CAR-T cell therapy (CART19/20) in patients with non-Hodgkin lymphoma, chronic lymphocytic leukemia, and small lymphocytic lymphoma. (A) Schematic of CD19/CD20 bispecific CAR construct. (B) Schematic of phase-1 dose escalation trial design. (C) Consolidated Standards of Reporting Trials (CONSORT) diagram of CART19/20 trial.

FIG. 3A-I. CART19/20 cells manufactured from naïve/memory T cells are enriched in memory phenotype. (A) Fold expansion of cell product during ex vivo manufacturing. Cell counts were normalized to counts on the day of transduction (day 3). Data are shown with color coding by patient (left), by whether starting cell population underwent CD14/CD25 depletion (middle), and by disease indication (right). (B-D) Flow cytometry performed on cryopreserved cell aliquots post thaw to characterize (B) CD3⁺ purity and transduction efficiency of final cell product, (C) T-cell subtype distribution among all CD3⁺ T cells, (D) T-cell subtype distribution among CAR-expressing T cells, and (E) % CD4⁺ among total T cells and CAR-expressing T cells. Te/exh: effector/exhausted T cells, CD45RA⁺/CD45RO⁻/CD62L⁻; Tem: effector-memory T cells, CD45RA⁻/CD45RO⁺/CD62L⁻; Tcm: central-memory T cells: CD45RA⁻/CD45RO⁺/CD62L⁺; naïve: CD45RA⁺/CD45RO⁻/CD62L⁺. (F) Phenotype of leukopak content prior to cell isolation. (G) Phenotype of cells obtained after isolation. (H) CD14 and CD25 expression patterns among CD62L+ cells in patient leukopak content prior to cell isolation. (I) CD3 and CD62L expression patterns among cells obtained after isolation. In panels B-G and I, red underscoring of patient ID indicates products that did not undergo CD14/CD25 depletion.

FIG. 4. Patients refractory to multiple prior lines of treatment respond to CART19/20 cell therapy. Timeline of individual patients' response to prior treatment and to CART19/20 cell therapy. The disease indication and dose of CART19/20 cells received are also indicated for each patient. MCL, mantle-cell lymphoma; FL, follicular lymphoma; PMBCL, primary mediastinal B-cell lymphoma; DLBCL, diffuse large B-cell lymphoma; tFL, transformed follicular lymphoma.

FIG. 5A-G. CART19/20 cell therapy is highly effective in treating relapsed/refractory non-Hodgkin lymphomas. (A) Representative PET scans of patients treated with CART19/20. (B) Overall survival and progression-free survival curves from the time of CART19/20 cell infusion. (C) PET scan obtained at screening for Patient 003, indicating pulmonary involvement of PMBCL. (D) Immunohistochemistry (IHC) analysis of Patient 003 tissue biopsies; original magnification ×160. Supraclavicular lymph node biopsy obtained at screening and bone-marrow biopsy obtained 14 days post CART19/20 infusion were analyzed by IHC. Results reveal rapid emergence of a CD19⁻CD20⁻BCL6⁻cMYC⁻ tumor population within 14 days of CART19/20 treatment. (E-G) C-reactive protein (CRP), ferritin, and lactate dehydrogenase (LDH) levels of all patients treated with CART19/20 cell therapy.

FIG. 6A-F. CART19/20 cells exhibit sustained persistence and efficacy with strong safety. (A) Flow cytometry analysis on peripheral blood samples collected from Patient 004 at screening as well as 7- and 14-days post CART19/20 infusion. CD19 and CD20 surface staining results indicate the presence of CD20⁺CD19^{dim/-} cells in Patient 004 prior to CART19/20 cell treatment. (B) PET scans of Patient 004 at the time of relapse and at 60 days post second dose of CART19/20 cells. (C) Presence of CAR transgene as quantified by droplet digital PCR. The psi signal integrated through lentiviral transduction was quantified. Inset shows zoomed-in data from the first 30 days post CART19/20 infusion. (D) Presence of CAR-expressing T cells among peripheral blood mononuclear cells (PBMCs) as quantified by flow cytometry. (E) Presence of CAR⁺ cells among T cells in patient peripheral blood as quantified by flow cytometry. (F) Presence of CD19⁺ and/or CD20⁺ cells among lymphocytes as quantified by flow cytometry. A 3% line is shown to denote threshold for B-cell aplasia.

FIG. 7A-B. CART19/20 cell product characteristics. (A) T-cell subtype distribution in cryopreserved CART19/20 cell products, with data segregated for CD4⁺ and CD8⁺ populations. Results indicate CD4⁺ T cells are more enriched in the central-memory phenotype compared to CD8⁺ T cells. Te/exh: effector/exhausted T cells, CD45RA⁺/CD45RO⁻/CD62L⁻; Tem: effector-memory T cells, CD45RA⁻/CD45RO⁺/CD62L⁻; Tcm: central-memory T cells: CD45RA⁻/CD45RO⁺/CD62L⁺; naïve: CD45RA⁺/CD45RO⁻/CD62L⁺. (B) Viability of cell product during ex vivo manufacturing. Data are shown with color coding by patient (left), by whether starting cell population underwent CD14/CD25 depletion (middle), and by disease indication

FIG. 8. CD14/CD20 depletion did not significantly alter regulatory T (Treg) cell content in CART19/20 cell product and in patient peripheral blood post infusion. Intracellular staining of starting (unactivated) T_{N/MEM} population, CART19/20 final product, and patient PBMC collected 14 days post CART19/20 infusion. FOXP3⁺ frequency is shown as a percentage of CD3+CD4+ T cells.

FIG. 9A-C. Patient 009 exhibited elevated cytokine, C-reactive protein (CRP), and ferritin levels prior to and after CART19/20 cell infusion. (A) Serum levels of various cytokines measured by Luminex multiplex assay. (B) CRP levels for Patient 009 (left) and all patients (right). (C) Ferritin levels for Patient 009 (left) and all patients (right).

FIG. 10. PET scans taken for all patients treated with CART19/20 cell therapy.

FIG. 11A-B. S5. T cell populations become CD8-dominant post CART19/20 cell infusion. % CD8⁺ among (A) CAR-expressing T cells and (B) all CD3⁺ T cells was quantified by flow cytometry. Data are shown for CAR-T cells only up to day 90 post infusion as values post day 90 become unreliable due to low CAR⁺ cell count detected by flow. FP: final product (i.e., cryopreserved CART19/20 cells).

### DETAILED DESCRIPTION

### I. Administration of Therapeutic Compositions

The therapeutic cells of the disclosure may be administered by any route of administration. In some aspects, the cells are administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The cells can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### II. Cellular Therapies

### A. Cell Culture

In some aspects, cells may be cultured for at least between about 10 days and about 40 days, for at least between about 15 days and about 35 days, for at least between about 15 days and 21 days, such as for at least about 15, 16, 17, 18, 19 or 21 days. In some aspects, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Licbovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some aspects, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other aspects may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular aspects, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular aspects of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain aspects cells are introduced into a culture system that features at least one substrate surface that is generally compatible with adherence of cells thereto, such that the plated cells can contact the said substrate surface, such aspects encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain aspects, the marker proteins used to identify and characterize the cells are selected from the list consisting of c-Kit. Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### C. Pharmaceutical Compositions

In certain aspects, the compositions or agents for use in the methods, such as the cell compositions, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### D. Adoptive Cell Therapy

Adoptive Cell Therapy is a form of passive immunization by the transfusion (adoptive cell transfer) of immune cells, in particular T-cells. T cells are found in blood and tissue and usually activate when they find foreign pathogens or other antigens that T-cell's surface receptors encounter parts of foreign proteins (antigens) that are displayed on surface of other cells. These latter cells can be either infected cells, or antigen presenting cells (APCs) that are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the expansion and the reinfusion of the resulting cells. Activation can take place through gene therapy, or by exposing the T cells to tumor antigens. Additional details on the preparation, selection, use, combination with other therapies, an/or administration of cells for ACT treatment are described in the literature (Cook K et al., 2018, Elahi R et al., 2018; Sharma P. et al., 2017).

In some aspects, the adoptive cell therapy comprises dendritic cell therapy, which provokes anti-tumor responses by causing dendritic cells to present tumor antigens to lymphocytes, and then activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T. One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF. Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies may include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

In some aspects, the adoptive cell therapy comprises CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy. Exemplary CAR-T therapies include Tisagenlecleucel (Kymriah) and Axicabtagene ciloleucel. In some aspects, the CAR-T therapy targets CD19 or CD20.

### E. Chimeric Antigen Receptors

### 1. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some aspects, a signal peptide directs the nascent protein into the endoplasmic reticulum. This is essential if a receptor is to be glycosylated and anchored in the cell membrane. Generally, the signal peptide natively attached to the amino-terminal most component is used (e.g., in an scFv with orientation light chain-linker-heavy chain, the native signal of the light-chain is used).

In some aspects, the signal peptide is cleaved after passage of the endoplasmic reticulum (ER), i.e., is a cleavable signal peptide. In some aspects, a restriction site is at the carboxy end of the signal peptide to facilitate cleavage.

### 2. Antigen Binding Domain

Polypeptides of the present disclosure may comprise one or more antigen binding domains. An “antigen binding domain” describes a region of a polypeptide capable of binding to an antigen under appropriate conditions. In some aspects, an antigen binding domain is a single-chain variable fragment (scFv) based on one or more antibodies (e.g., CD20 antibodies). In some aspects, an antigen binding domain comprise a variable heavy (VH) region and a variable light (VL) region, with the VH and VL regions being on the same polypeptide. In some aspects, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1. CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M. 10⁻⁹M, 10⁻¹⁰M, 10⁻¹¹M, 10⁻¹²M, or 10⁻¹³M. In some aspects, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some aspects, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some aspects, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some aspects, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### 3. Peptide Spacer

A peptide spacer, such as an extracellular spacer may link an antigen-binding domain to a transmembrane domain. In some aspects, a peptide spacer is flexible enough to allow the antigen-binding domain to orient in different directions to facilitate antigen binding. In one embodiment, the spacer comprises the hinge region from IgG. In some aspects, the spacer comprises or further comprises the CH2CH3 region of immunoglobulin and portions of CD3. In some aspects, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some aspects, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some aspects, the extracellular spacer consists of or comprises a hinge region from an immunoglobulin (e.g., IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer may have effects on the CAR's signaling activity and/or the CAR-T cells' expansion properties in response to antigen-stimulated CAR signaling. In some aspects, a shorter spacer such as less than 50, 45, 40, 30, 35, 30, 25, 20, 15, 14, 13, 12, 11, or 10 amino acids is used. In some aspects, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some aspects, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### 4. Transmembrane Domain

Polypeptides of the present disclosure may comprise a transmembrane domain. In some aspects, a transmembrane domain is a hydrophobic alpha helix that spans the membrane. Different transmembrane domains may result in different receptor stability.

In some aspects, the transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions.

Any transmembrane domain that provides for insertion of a polypeptide into the cell membrane of a eukaryotic (e.g., mammalian) cell may be suitable for use. In some aspects, the transmembrane domain is derived from CD28, CD8, CD4, CD3-zeta, CD134, or CD7.

### 5. Cytoplasmic Region

After antigen recognition, receptors of the present disclosure may cluster and a signal transmitted to the cell through the cytoplasmic region. In some aspects, the costimulatory domains described herein are part of the cytoplasmic region. In some aspects, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2 (L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2 (L/I)) (X3) n (YX1X2 (L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some aspects, a suitable cytoplasmic region can comprise an ITAM motif-containing portion of the full length DAP12 amino acid sequence. In some aspects, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). In some aspects, a suitable cytoplasmic region can comprise an ITAM motif-containing portion of the full length FCER1G amino acid sequence.

In some aspects, the cytoplasmic region is derived from T cell surface glycoprotein CD3 delta chain (also known as CD3D; CD3-DELTA; T3D; CD3 antigen, delta subunit; CD3 delta; CD3□; CD3d antigen, delta polypeptide (TiT3 complex); OKT3, delta chain; T cell receptor T3 delta chain; T cell surface glycoprotein CD3 delta chain; etc.). In some aspects, a suitable cytoplasmic region can comprise an ITAM motif-containing portion of the full length CD3 delta amino acid sequence. In some aspects, the cytoplasmic region is derived from T cell surface glycoprotein CD3 epsilon chain (also known as CD3e, CD3□; T cell surface antigen T3/Leu-4 epsilon chain, T cell surface glycoprotein CD3 epsilon chain, AI504783, CD3, CD3-epsilon, T3e, etc.). In some aspects, a suitable cytoplasmic region can comprise an ITAM motif-containing portion of the full length CD3 epsilon amino acid sequence. In some aspects, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some aspects, a suitable cytoplasmic region can comprise an ITAM motif-containing portion of the full length CD3 gamma amino acid sequence. In some aspects, the cytoplasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). In some aspects, a suitable cytoplasmic region can comprise an ITAM motif-containing portion of the full length CD3 zeta amino acid sequence.

In some aspects, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some aspects, a suitable cytoplasmic region can comprise an ITAM motif-containing portion of the full length CD79A amino acid sequence.

### 6. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some aspects, the costimulatory region is derived from an intracellular portion of the transmembrane protein 4-1BB (also known as TNFRSF9; CD137; CDw137; ILA; etc.). In some aspects, the costimulatory region is derived from an intracellular portion of the transmembrane protein CD28 (also known as Tp44). In some aspects, the costimulatory region is derived from an intracellular portion of the transmembrane protein ICOS (also known as AILIM, CD278, and CVID1). In some aspects, the costimulatory region is derived from an intracellular portion of the transmembrane protein OX-40 (also known as TNFRSF4, RP5-902P8.3, ACT35, CD134, OX40, TXGP1L). In some aspects, the costimulatory region is derived from an intracellular portion of the transmembrane protein BTLA (also known as BTLA1 and CD272). In some aspects, the costimulatory region is derived from an intracellular portion of the transmembrane protein CD27 (also known as S 152, T14, TNFRSF7, and Tp55). In some aspects, the costimulatory region is derived from an intracellular portion of the transmembrane protein CD30 (also known as TNFRSF8, D1S166E, and Ki-1). In some aspects, the costimulatory region is derived from an intracellular portion of the transmembrane protein GITR (also known as TNFRSF18, RP5-902P8.2, AITR, CD357, and GITR-D). In some aspects, the costimulatory region derived from an intracellular portion of the transmembrane protein HVEM (also known as TNFRSF14, RP3-395M20.6, ATAR, CD270, HVEA, HVEM, LIGHTR, and TR2).

### Detection Peptides

In some aspects, the polypeptides described herein may further comprise a detection peptide. Suitable detection peptides include hemagglutinin (HA; e.g., YPYDVPDYA (SEQ ID NO: 4); FLAG (e.g., DYKDDDDK (SEQ ID NO:5); c-myc (e.g., EQKLISEEDL; SEQ ID NO:6), and the like. Other suitable detection peptides are known in the art.

### 7. Peptide Linkers

In some aspects, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G) n, glycine-serine polymers (including, for example, (GS)n, (GSGGS)n (SEQ ID NO:7), (G4S) n and (GGGS)n (SEQ ID NO: 8), where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO: 9), GGSGG (SEQ ID NO:10), GSGSG (SEQ ID NO:11), GSGGG (SEQ ID NO:12), GGGSG (SEQ ID NO:13), or GSSSG (SEQ ID NO:14).

### F. Cells

Certain aspects relate to cells comprising polypeptides or nucleic acids of the disclosure. In some aspects the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### III. Nucleic Acids and Polypeptides

### A. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### 1. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### 2. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### 3. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### B. Polypeptides

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence/peptide has been removed. The protein may be isolated directly from the organism of which it is native, produced by recombinant DNA/exogenous expression methods, or produced by solid phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of one of SEQ ID NOS: 1-14. In specific aspects, the peptide or polypeptide is or is based on a human sequence. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS: 1-14 (or any derivable range therein) and may be a substitution with any amino acid or may be a substitution with a alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-14.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-14 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS: 1-14.

In some aspects, the protein, polypeptide, or nucleic acid may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS: 1-14.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS: 1-14 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS: 1-14.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS: 1-14 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS: 1-14.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within +2 is included. In some aspects of the invention, those that are within +1 are included, and in other aspects of the invention, those within +0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### IV. Additional Therapies

### A. Immunotherapy

In some aspects, the methods comprise administration of an additional therapy. In some aspects, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Checkpoint Inhibitors and Combination Treatment

Aspects of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### B. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### D. Surgery

In some aspects, the additional therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### V. Sequences

| | | SEQ |
|---|---|---|
| | | ID |
| Description | Sequence | NO: |
| CD20 scFv (Vl/Vh)- | METDTLLLWVLLLWVPGSTGDIVLTQSPAILSASPGEK | 1 |
| (G4S)₄ linker-CD19 | VTMTCRASSSVNYMDWYQKKPGSSPKPWIYATSNLAS | |
| scFv (Vl/Vh)-IgG4 | GVPARFSGSGSGTSYSLTISRVEAEDAATYYCQQWSFN | |
| Hinge-CD28tm-4- | PPTFGGGTKLEIKGSTSGGGSGGGSGGGGSSEVQLQQS | |
| 1BB-zeta | GAELVKPGASVKMSCKASGYTFTSYNMHWVKQTPGQ | |
| | GLEWIGAIYPGNGDTSYNQKFKGKATLTADKSSSTAY | |
| | MQLSSLTSEDSADYYCARSNYYGSSYWFFDVWGAGT | |
| | TVTVSSGGGGSGGGGSGGGGSGGGGSEVKLQESGPGL | |
| | VAPSQSLSVTCTVSGVSLPDYGVSWIRQPPRKGLEWLG | |
| | VIWGSETTYYNSALKSRLTIIKDNSKSQVFLKMNSLQT | |
| | DDTAIYYCAKHYYYGGSYAMDYWGQGTSVTVSSGST | |
| | SGSGKPGSGEGSTKGDIQMTQTTSSLSASLGDRVTISCR | |
| | ASQDISKYLNWYQQKPDGTVKLLIYHTSRLHSGVPSRF | |
| | SGSGSGTDYSLTISNLEQEDIATYFCQQGNTLPYTFGGG | |
| | TKLEITESKYGPPCPPCPMFWVLVVVGGVLACYSLLVT | |
| | VAFIIFWVKRGRKKLLYIFKQPFMRPVQTTQEEDGCSC | |
| | RFPEEEEGGCELRVKFSRSADAPAYQQGQNQLYNELN | |
| | LGRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPRLEGGGEGRGSLLTCGDVEENPG | |
| | PRMLLLVTSLLLCELPHPAFLLIPRKVCNGIGIGEFKDSL | |
| | SINATNIKHFKNCTSISGDLHILPVAFRGDSFTHTPPLDP | |
| | QELDILKTVKEITGFLLIQAWPENRTDLHAFENLEIIRGR | |
| | TKQHGQFSLAVVSLNITSLGLRSLKEISDGDVIISGNKN | |
| | LCYANTINWKKLFGTSGQKTKIISNRGENSCKATGQVC | |
| | HALCSPEGCWGPEPRDCVSCRNVSRGRECVDKCNLLE | |
| | GEPREFVENSECIQCHPECLPQAMNITCTGRGPDNCIQC | |
| | AHYIDGPHCVKTCPAGVMGENNTLVWKYADAGHVC | |
| | HLCHPNCTYGCTGPGLEGCPTNGPKIPSIATGMVGALL | |
| | LLLVVALGIGLFM* | |
| CD20 scFv (Vl/Vh)- | DIVLTQSPAILSASPGEKVTMTCRASSSVNYMDWYQK | 3 |
| (G4S)₄ linker-CD19 | KPGSSPKPWIYATSNLASGVPARFSGSGSGTSYSLTISR | |
| scFv (Vl/Vh) | VEAEDAATYYCQQWSFNPPTFGGGTKLEIKGSTSGGG | |
| | SGGGSGGGGSSEVQLQQSGAELVKPGASVKMSCKASG | |
| | YTFTSYNMHWVKQTPGQGLEWIGAIYPGNGDTSYNQ | |
| | KFKGKATLTADKSSSTAYMQLSSLTSEDSADYYCARS | |
| | NYYGSSYWFFDVWGAGTTVTVSSGGGGSGGGGSGGG | |
| | GSGGGGSEVKLQESGPGLVAPSQSLSVTCTVSGVSLPD | |
| | YGVSWIRQPPRKGLEWLGVIWGSETTYYNSALKSRLTI | |
| | IKDNSKSQVFLKMNSLQTDDTAIYYCAKHYYYGGSYA | |
| | MDYWGQGTSVTVSSGSTSGSGKPGSGEGSTKGDIQMT | |
| | QTTSSLSASLGDRVTISCRASQDISKYLNWYQQKPDGT | |
| | VKLLIYHTSRLHSGVPSRFSGSGSGTDYSLTISNLEQEDI | |
| | ATYFCQQGNTLPYTFGGGTKLEIT | |
| Murine kappa chain | METDTLLLWVLLLWVPGSTG | 2 |
| signal peptide | | |

### VI. Examples

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: Anti-CD19/CD20 Bispecific Chimeric Antigen Receptor (CAR) in Naïve/Memory T-Cells for Relapsed or Refractory B-Cell Lymphomas

Introduction: Despite excellent responses to anti-CD19 CAR T-cell therapy, 50% of patients with relapsed/refractory (R/R) B-cell lymphomas (BCL) eventually relapse primarily due to poor CAR-T persistence or CD19 antigen escape. Preclinical data demonstrate that engineering of bispecific anti-CD19/CD20 CAR in naïve/memory T-cells (TN/MEM) via lentiviral transduction effectively targets tumor cells, overcomes antigen escape and enhances CART persistence (Zah E et al., Cancer Immunol Res, 2016).

Objectives: The inventors are evaluating safety and early efficacy outcomes of a first-in-human phase 1 clinical trial with bispecific CD19/CD20 CAR T-cells for patients with R/R BCL (NCT04007029).

Methods: Patients with measurable disease after 2 or more lines of therapy for diffuse large B-cell lymphoma (DLBCL) and primary mediastinal B-cell lymphoma (PMBCL), and 3 or more lines of therapy for mantle cell lymphoma (MCL), follicular lymphoma (FL) and chronic lymphocytic leukemia (CLL) were included. Autologous leukocytes obtained by leukapheresis were sorted for CD14−/CD25−/CD62L+TN/MEM cells, followed by lentiviral transduction of the bispecific CD19/CD20 CAR. Following Cy-Flu lymphodepletion, patients are infused with CD19/CD20 CAR TN/MEM cells, at doses ranging from 5×10⁷ and 2×10⁸ CAR positive cells.

Results: To date, 8 patients are enrolled in the trial and 7 have received the anti-CD19/CD20 CAR T-cells. 5 patients received 5×10⁷ CAR positive cells and 2 patients received 2×10⁸ CAR positive cells. All patients had CD19 and CD20 positivity confirmed on tissue biopsy prior to enrollment. Patients previously received a median of 3 prior lines of therapy, and 6 patients received bridging therapy. Six patients developed cytokine release syndrome (CRS), all grade 1, and no patient had immune effector cell-associated neurotoxicity syndrome (ICANS). Peak expansion was noted on day 14. Median follow up is 10.0 months, with 6 patients in ongoing complete remission. The 1 patient who did not respond had early disease progression, with CD19 and CD20 negative mediastinal lymphoma at day 14 after CAR infusion. Median progression-free survival (PFS) and overall survival (OS) were not reached, and all responders demonstrate ongoing CAR T-cell persistence and B-cell aplasia by data cutoff.

Conclusion: Bispecific CD19/CD20 CAR in naïve/memory T-cells were safe and effective in patients with R/R BCL. Although further enrollment and follow-up is necessary to confirm these early promising results, the inventors are encouraged that the modification of naïve/memory T-cells and dual antigen targeting could potentially obviate the current challenges of poor CAR-T persistence and antigen escape, respectively. The trial is currently actively enrolling.

### Example 2: Manufacturing of CD19/CD20 CAR-T Cells

On Day 0, a patient leukapheresis product containing 8.58×10⁹ white blood cells was analyzed by flow cytometry and found to contain 8.8% CD3+ and 8.9% CD62L+ cells. Among the CD62L+ cells, 15.67% of cells were CD14+ and/or CD25+. The leukapheresis bag was stored on a rotator in ambient air for 18.8 hours. The next morning (Day 1), the leukapheresis product was transferred to a sterile Hamlett tube and washed with labeling buffer (CliniMACS PBS-EDTA supplemented with human serum albumin). Washed cells are resuspended in labeling buffer and incubated with anti-CD14 and anti-CD25 magnetic microbeads. The labeling reaction was allowed to proceed at room temperature for 30 minutes with gentle agitation on a rotator. Labeled cells were centrifuged and resuspended in fresh labeling buffer, and processed through a depletion program on the CliniMACS. After depletion, 3.23×10⁹ cells at 99% viability were collected in the CD14−/CD25− fraction. These cells were washed with labeling buffer. Washed cells were resuspended in labeling buffer and treated with GammaGard to block non-specific labeling. After a 10-minute incubation on an orbital rotator, anti-CD62L magnetic microbeads were added to the cells, which were then incubated at room temperature for 30 minutes with gentle agitation on a rotator. Labeled cells were centrifuged and resuspended in fresh labeling buffer, and processed through an enrichment program on the CliniMACS. After enrichment, 1.438×10⁹ cells at 97% viability were collected in the CD62L+ fraction. A sample was taken for sterility testing. An aliquot of this final isolation product, termed T_{N/MEM} cells, was analyzed by flow cytometry and found to be 88% CD3+ and 88% CD62L+.

Eight hundred million T_{N/MEM} cells were resuspended in CTS OpTimizer Basal Media, and activated with MACS GMP T-cell TransAct at 1:35 dilution, together with 50 IU/mL interleukin (IL)-2 and 0.5 ng/mL IL-15. Cells were transferred to a VueLife bag and kept in 5% CO₂ incubator at 37° C. After a 40-hour incubation (Day 3), 6.24×10⁸ cells at 90% were present in the bag. Three-hundred and eighty million cells were resuspended in 380 mL and transduced with lentivirus at an MOI of 1.5 in the presence of 25 μg/mL protamine sulfate, IL-2, and IL-15. Cells were kept in 5% CO₂ incubator at 37° C. for 6 hours, and then further diluted with 380 mL of media. On Day 5, cells were diluted 1-to-3 with fresh media and fed with IL-2 and IL-15. On Day 7, cells were centrifuged to remove TransAct-containing media and resuspended in 200 mL fresh media. Cells were counted and 9.32×10⁸ cells at 90% viability were present. Cell suspension was diluted to 0.45×10⁶ cells/mL with fresh culture media supplemented with IL-2 and IL-15. Cell samples were taken for replication-competent lentivirus (RCL) and sterility testing. On Day 10, 1.533×10⁹ cells at 96% viability were present. Cells were diluted to 0.6×10⁶ cells/mL with fresh culture media supplemented with IL-2 and IL-15. On Day 11, 1.661×10⁹ cells at 97% viability were present. Cells were diluted to 0.6×10⁶ cells/mL with fresh culture media supplemented with IL-2 and IL-15. A cell sample was taken for flow cytometry analysis, with results indicating 99% CD3+ and 56% CAR+ expression.

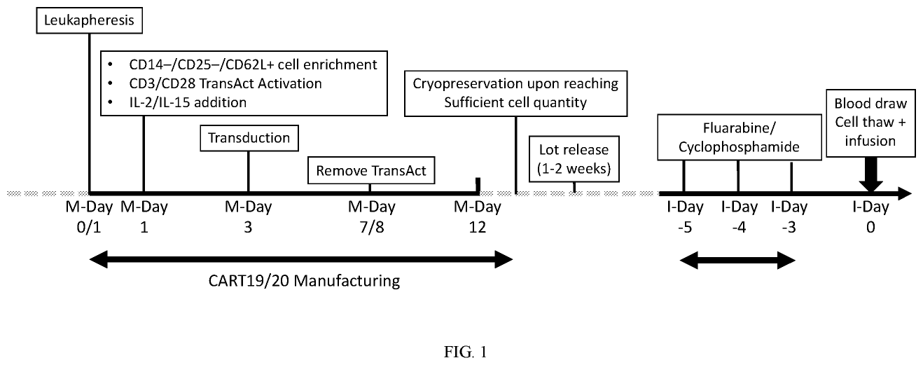

On Day 12, 1.605×10⁹ cells at 95% viability were present. A supernatant sample was taken for mycoplasma testing, and a cell sample was taken for RCL testing. Cells were centrifuged and washed with Isolyte. Post wash, cells were resuspended in 400 mL Isolyte and counted, revealing 1.552×10⁹ cells at 95% viability. Cells were filtered through a 40-micron filter, centrifuged, and resuspended in 103.5 mL of CryoStor CS5 for a final concentration of 15×10⁶ cells/mL. Cells were aliquoted into CryoMACS freezing bags and cryovials, and subsequently transferred into a CryoMed Controlled Rate Freezer for cryopreservation. Aliquots were subsequently thawed for quality-control (QC) release testing. The thawed product was 84% viable, 98% CD3+, and 50% CAR+. The product passed all QC testing, including sterility, mycoplasma, endotoxin, and RCL. Each infusion bag contained 1.65×10⁸ CAR+ T cells, meeting the desired dose of 2×10⁸±30% CAR+ T cells. FIG. 1 provides an overview of the manufacturing process.

### Example 3: Comparison of Similar CAR Technologies

This example provides a comparison between clinical data obtained using the inventor's CAR-T cells to published data of similar CAR-T cells. The comparison demonstrates the unexpected superior therapeutic efficacy and reduced toxicity of the manufactured CAR T cells of the current application in comparison with the similar constructs.

The CD19/CD20 bispecific chimeric antigen receptor (CAR) made by the process described in the application entered clinical testing in the context of CAR-T cell therapy in the fall of 2019, for the treatment of non-Hodgkin B-cell lymphoma. Since November 2019, at least ten patients have been treated and evaluated for response.

Among the ten patients evaluated for response to date (Jul. 11, 2022), nine responded to therapy, yielding a 90% overall response rate (ORR). Among these, seven reached complete remission (CR), yielding an 70% (7/10) CR rate. Each patient received a single dose of either 50 million±30% (cohort 1; 5 patients) or 200 million±30% (cohort 2; 2 patients) CAR-expressing T cells. The dosage level was fixed and not adjusted by patient weight. CR rates to date are 57% (4/7) for cohort 1 and 100% (3/3) for cohort 2. This is summarized in Table 3 under the “UCLA” row.

Among the seven patients treated to date (July 2022), none has experienced any neurotoxicity, and none has experienced cytokine release syndrome (CRS) above grade 1 (i.e., the lowest level of severity). Therefore, the inventor's trial demonstrates that the CD19/CD20 CAR-T cell therapy described in the application is highly efficacious and safe for treating human patients. This result is in stark contrast with other bispecific CAR-T cell therapies that have been evaluated in the clinic. For example, Lentigen has conducted a similar phase-1 dose-escalation trial at the Medical College of Wisconsin to evaluate a CD19/CD20 CAR (relating to U.S. Pat. No. 10,442,867) similar to the one for which the current application describes. According to their published results, the Lentigen CAR-T cell therapy yielded 33% (1/3) CR rate at the 0.75 million/kg dose level (comparable to the inventors' cohort 1, which corresponds to 0.71 million/kg when assuming an average 70-kg adult patient). To reach a 75% (12/16) CR rate, Lentigen had to increase the dose to 2.5 million/kg, which is comparable to the inventors' cohort 2 level. At this higher dosage level, Lentigen reported a patient with grade-4 CRS and grade-4 neurotoxicity. Lentigen's overall CR rate was 64% (14/22), with multiple cases of ≥3 CRS and/or neurotoxicity. Furthermore, the inventors' product is cryopreserved, whereas Lentigen used either cryopreserved or fresh (never frozen) cell products. Among patients treated with Lentigen's cryopreserved products, the overall CR rate was only 29% (2/7). The result's from Lentigen's publication are summarized in Table 3 under the “Lentigen” row. Therefore, compared to Lentigen's construct, the inventors' CD19/CD20 CAR has exhibited greatly superior efficacy as well as safety profile.

For additional comparison, another CD19/CD20 CAR-T cell therapy has been evaluated at the Chinese PLA General Hospital. This comparison is summarized in Table 3. Their reported data showed an overall CR rate of 70% (60/87), with doses ranging from 0.50 million/kg to 8 million/kg. Importantly, 85 of the 87 patients received doses of ≥1.8 million cells/kg, which is more than twice the dose compared to cohort 1 in the inventors' trial. Their trial also observed substantial toxicity, including 3 deaths due to pulmonary infection or injury, and multiple cases of neurotoxicity and grade-3/4 CRS. Therefore, the inventors' trial results from CAR-T cells manufactured by the methods of the current application have proven superior compared to this set of clinical data in both safety and efficacy levels.

### Example 4: CD19/CD20 Bispecific Chimeric Antigen Receptor (CAR) in Naïve/Memory T Cells for the Treatment of Relapsed or Refractory Non-Hodgkin Lymphoma

To address antigen escape and loss of T-cell functionality, the inventors performed a phase-1 clinical trial (NCT04007029) evaluating autologous naïve and memory T (T_{N/MEM}) cells engineered to express a bispecific anti-CD19/CD20 CAR (CART19/20) for patients with relapsed/refractory NHL, with safety as the primary end point. Ten patients were treated with 36-165×10⁶ CART19/20 cells. No patient experienced neurotoxicity of any grade, or over grade-1 cytokine release syndrome. One case of dose-limiting toxicity (persistent cytopenia) was observed. Nine of ten patients achieved objective response (90% ORR), with seven achieving complete remission (70% CR rate). One patient relapsed after 18 months in CR, but returned to CR after receiving a second dose of CART19/20 cells. Median progression-free survival and overall survival were not reached with a 17-month median follow-up. In conclusion, CART19/20 T_{N/MEM} cells are safe and effective in patients with relapsed/refractory NHL, with durable responses achieved at low dosage levels.

Effective chimeric antigen receptor (CAR)-T cell therapy exerts a strong selective pressure against cancer cells that express the CAR-targeted antigen, and downregulation or loss of expression is the natural escape route for target antigens that are not critical to cell survival. Accurate quantification of relapse rate attributable to CD19 antigen escape is complicated by lack of tissue acquisition following relapse, and reported CD19-negative relapse frequencies range from 27% to 100% of relapsed cases among patients with leukemia and lymphoma (1-5). The frequency of cases with CD19-negative relapse demonstrates the susceptibility of CD19 to antigen loss, and points to the identification of alternative target antigens that are more resistant to gene-expression downregulation as a potential remedy.

To address the problem of CD19 antigen escape, the inventors developed a CD19/CD20 bispecific CAR-T cell therapy, and previously demonstrated its ability to eradicate B-cell lymphoma with heterogenous CD19 expression and prevent relapse in mouse models of human lymphoma (6,7). CD19/CD20 bispecific CAR-T cells outperformed single-input CD19 CAR-T cells in achieving long-term, progression-free survival in a lymphoma xenograft model (6,7). CD20, like CD19, is pan-B-cell marker, and the first-line therapy for B-cell malignancies typically includes an anti-CD20 antibody such as rituximab (8). In fact, rituximab is commonly included in each subsequent line of chemotherapy administered to patients with NHL, yet CD20 antigen loss is a low-frequency event despite repeated cycles of CD20-targeted therapies (9), suggesting CD20 may be a suitable CAR target with low propensity for antigen escape. However, the clinical outcomes of CD20 CAR-T cell therapy have been uneven to date (10-13), resulting in more limited clinical advancement compared to CD19 CAR-T cell therapy. The inventors hypothesized that simultaneously targeting CD19 and CD20 would both enable high initial response rate and increased resistance to antigen escape. Importantly, dual targeting of CD19 and CD20 would not increase on-target, off-tumor toxicity compared to either CD19 or CD20 single-input CAR-T cell therapy because both CD19 and CD20 are B-cell-specific markers, thus limiting the off-tumor toxicity to healthy B cells whose aplasia is a clinically manageable condition (14).

### A. Results

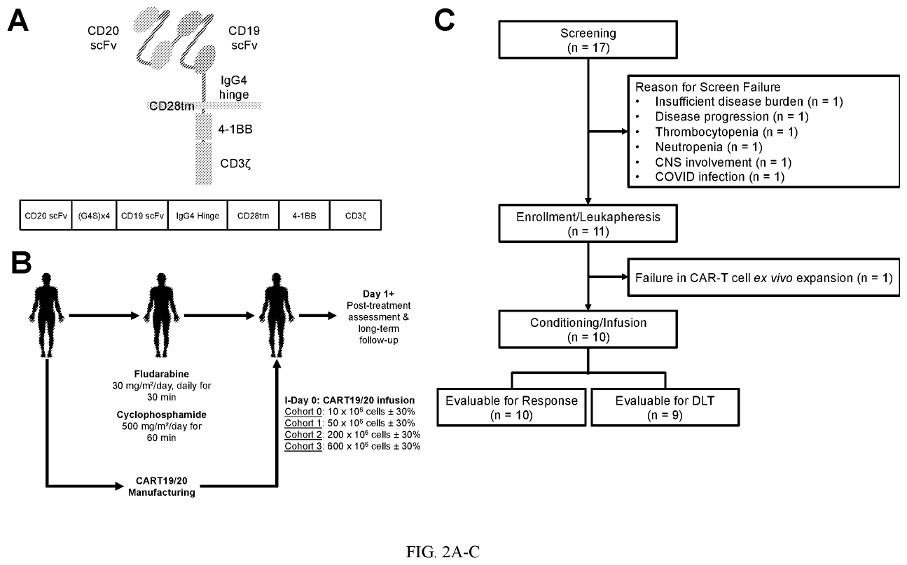

CAR construct and clinical trial design. The inventors generated a CD19/CD20 bispecific CAR consisting of a single-chain variable fragment (scFv) derived from the anti-CD20 monoclonal antibody Leu16 fused to a second scFv derived from the anti-CD19 monoclonal antibody FMC63, followed by fusion of the scFv domains to the hinge domain of human IgG4, the transmembrane domain of human CD28, and the cytoplasmic signaling domains of human 4-1BB and CD35 (FIG. 2A) (6,7). The bispecific CAR was encoded by a third-generation self-inactivating lentiviral vector under the control of an elongation factor 1 alpha (EF1-α) promoter (22). The inventors planned a phase-1 cell dose escalation trial with a fixed lymphodepletion chemotherapy of fludarabine 30 mg/m² daily for three days and cyclophosphamide 500 mg/m² daily for three days followed by CART19/20 infusion with dose levels of 50×10⁶ CAR+ T cells (DL1), 200×10⁶ CAR+ T cells (DL2), and 600×10⁶ CAR+ T cells (DL3), with each DL allowing ±30% range (FIG. 2B).

Patient characteristics. Seventeen patients were screened, 11 patients went onto leukapheresis (FIG. 2C), and ten patients received CART19/20 infusion in two cohorts (DL1, n=7; DL2, n=3) (FIG. 2C). The median age at the time of CART19/20 infusion was 59 (range, 29-70) (Table 1). The diagnoses were mantle-cell lymphoma (MCL; n=1), follicular lymphoma (FL; n=3), de novo diffuse large B-cell lymphoma (DLBCL; n=1), transformed FL to DLBCL (n=3), primary mediastinal B-cell lymphoma (PMBCL; n=1), and high-grade B-cell lymphoma (HGBCL; n=1) with BCL6 and cMYC double-hit rearrangement. All patients with FL had progression of disease within 24 months after front-line treatment (POD24). The median lines of prior therapy were 3.5 (range, 2-4). One patient (Patient 004) was refractory to prior anti-CD19 bispecific T-cell engager (BiTE) therapy. All patients were CAR naïve and had stage-4 disease at the time of CART19/20 treatment. Nine patients were given bridging therapy prior to infusion due to progressive disease (Table 1).

As of the data cutoff on Jul. 11, 2022, a total of ten patients were evaluable for response. Nine patients were evaluable for dose-limiting toxicity (DLT), including six treated at DL1 and three treated at DL2. A decision was made to not escalate to DL3 based on the strong efficacy outcomes observed at the two lower dosing levels. The maximum tolerated dose was not reached.

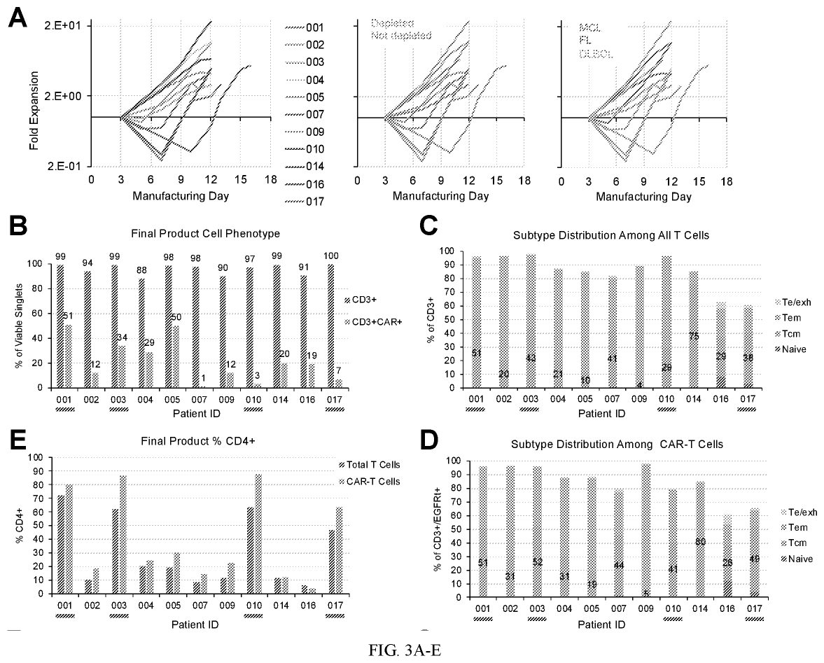

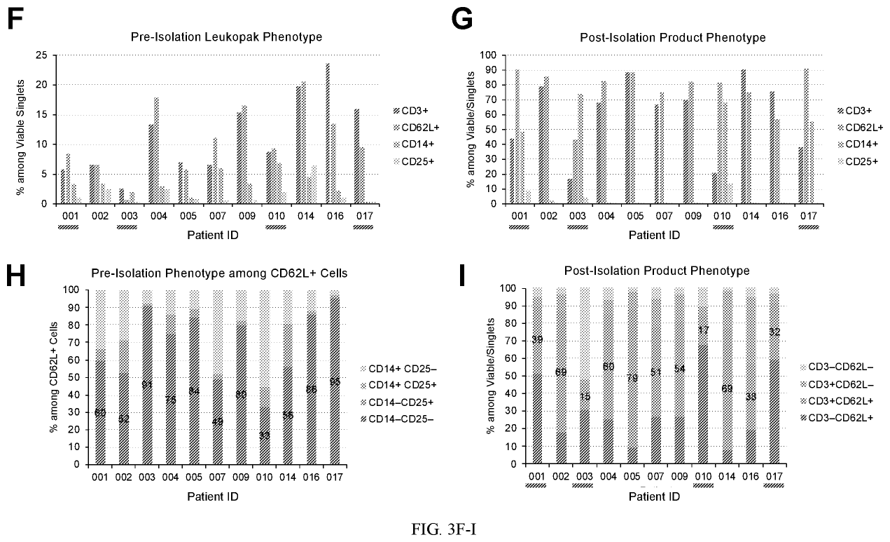

CART19/20 cell manufacturing and product characterization. Patient leukocytes harvested from leukapheresis were enriched for cells expressing CD62L, a marker for T_{N/MEM} cells, by magnetic bead-based cell separation. Leukapheresis products with greater than 5% CD14⁺ and/or CD25⁺ cells among viable singlets based on flow cytometry analysis were subjected to an additional CD14/CD25 depletion step to remove myeloid and regulatory T cells (Tregs), respectively, prior to CD62L enrichment. T_{N/MEM} cells were activated with an anti-CD3/CD28 colloidal nanomatrix-based activation reagent, lentivirally transduced to express the CD19/CD20 bispecific CAR, and expanded ex vivo for a total of 12 days (n=9), 14 days (n=1, Patient 016), or 16 days (n=1, Patient 007) prior to cryopreservation to yield the CART19/20 product. Ten of the 11 products manufactured met release criteria, with one manufacturing failure due to low CAR+ T-cell counts that did not meet dose requirements (Patient 007). Patient 007 was diagnosed with stage-4 DLBCL transformed from lymphoplasmatic lymphoma. The patient had a low absolute lymphocyte count (ALC) of 0.16×10³ cells/μL at the time of screening, and the patient's leukapheresis product was also low in white blood cell (WBC) count (32.79×10³/μL). Patient 007's cells transduced and expanded poorly during ex vivo manufacturing FIGS. 3A and B), resulting in failure to meet the required CART19/20 cell dose for infusion.

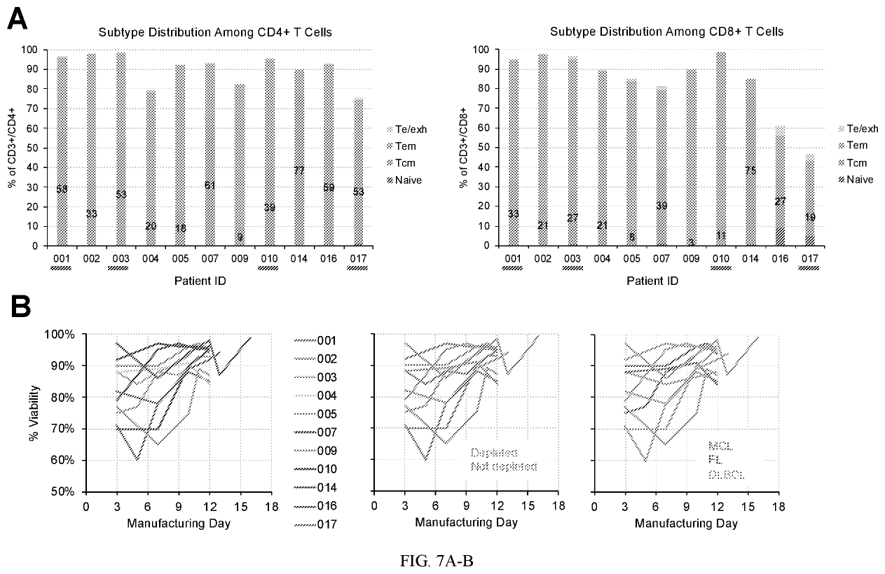

Overall, CART19/20 cell products contained a substantial fraction of central-memory T (Tcm) cells (median: 29.3%, range: 3.6%-74.9%; FIG. 3C), indicating the retention of memory phenotype in cell products manufactured from T_{N/MEM} cells. Of note, CAR-expressing T cells tend to have slightly higher Tem content (median: 40.9%, range: 5.3%-80.1%) compared to the overall T-cell population (FIG. 3D). A breakdown of CD4⁺ vs. CD8⁺ subtype distribution reveals that CAR⁺ T cells tend to have higher % CD4⁺ than the total T-cell population (FIG. 3E), and CD4⁺ T cells tend to be more enriched in the Tem phenotype compared to CD8⁺ T cells (FIG. 7A).

### 1. Depletion of CD14/CD25 Cells Results in CD8-Dominant T-Cell Products and No Significant Impact on Ex Vivo Cell Expansion, Transduction Efficiency, or Memory Phenotype Distribution.

The inventors chose to incorporate CD14 depletion in order to minimize the presence of myeloid cells, which had been reported to reduce T-cell activation through phagocytosis of activation agents (23) and could potentially reduce transduction efficiency by competing with T cells for lentivirus uptake. The removal of immunosuppressive Tregs through CD25 depletion (24) aimed to further enhance the anti-tumor efficacy of CART19/20 products. The minimum threshold of ≥5% CD14⁺ and/or CD25⁺ cells for depletion was based on the empirical observation that up to 5% of antigen-positive cells can remain even after depletion during preclinical process development. Following this criterion, the leukapheresis products of Patients 001 and 003 were not subjected to depletion and proceeded directly to CD62L enrichment (FIG. 3F). However, the post-isolation cell population for Patients 001 and 003 showed a notable increase in % CD14⁺ (from 3% and 2% to 48% and 74%, respectively), together with an uptick in % CD25⁺ (from 0.9% and 0.3% to 8% and 4%, respectively; FIG. 3G). This unintended enrichment is consistent with the fact that a large fraction of CD62L⁺ cells in patient leukapheresis products are CD14⁺ and/or CD25⁺ (FIG. 3H), thus the CD62L enrichment step would simultaneously result in selective retention of CD14⁺ and/or CD25⁺ cells. Furthermore, the adherent nature of myeloid cells may also facilitate the retention of CD14⁺ cells during bead-based cell sorting in the absence of a depletion step. Based on these observations, the protocol was amended to trigger CD14/CD25 depletion when ≥5% of CD62L⁺ cells (as opposed to ≥5% of viable singlets) were CD14⁺ and/or CD25⁺, starting with the product for Patient 004.

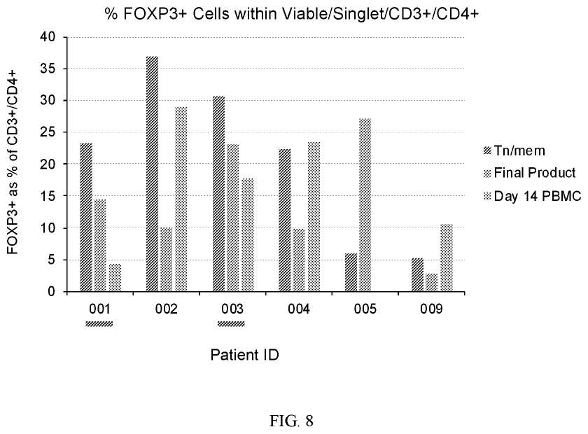

Despite the fact that the post-isolation cell population for patients 001 and 003 contained a substantial number of myeloid cells, the CART19/20 cultures for these two patients showed typical fold expansion and viability levels during the manufacturing process (FIG. 3A; FIG. 7B). Both final products exhibited high levels of CD3⁺ purity and CAR transduction efficiency (FIG. 3B), and had similar T-cell subtype distribution at the time of cryopreservation as products made from CD14/CD25-depleted cells (FIGS. 3C and D). The only clear difference was that products generated from non-depleted starting material were CD4 dominant, whereas products generated from CD14/CD25-depleted cells were CD8 dominant (FIG. 3E), potentially due to myeloid cells' ability to stimulate CD4⁺ cells through MHC-II antigen presentation. Taken together, these results indicate the culture conditions succeeded in selectively expanding T cells while avoiding the negative impacts of myeloid cells observed in the manufacturing processes described in previous reports (23,25,26). Given that the starting % CD25⁺ was <1% in both Patients 001 and 003, there was minimal concern regarding Treg enrichment. Indeed, FOXP3 intracellular staining showed no clear correlation between CD25 depletion or lack thereof with the level of FOXP3 expression among CD4⁺ T cells in CART19/20 final products (FIG. 8).

Among the subsequent products, two more were manufactured in the absence of CD14/CD25 depletion (Patients 010 and 017). Both products were enriched in CD4⁺ T cells, with no distinctive pattern in ex vivo expansion rate, transduction efficiency, or T-cell subtype distribution compared to CD14/CD25-depleted products (FIG. 3B-E), consistent with initial observations made with Patients 001 and 003.

### 2. Disease Histology as Risk Factor in CAR-T Cell Manufacturing.

Among the 11 manufacturing campaigns performed for this trial, three experienced a T-cell population contraction of >2 folds between the day of activation (day 1) and the day of transduction (day 3) (FIG. 3A). All three products were for patients diagnosed with DLBCL subtypes (Patients 007, 016, 017). Despite the reduced cell expansion, products for Patients 007, 016, and 017 had average T-cell subtype distribution (FIGS. 3C and D). Transduction efficiency was low for two of the three products (FIG. 3B), and the product for Patient 007 failed to meet the dose-level requirement as previously noted. However, DL1 products were successfully manufactured for Patients 016 and 017 and administered to the patients.

Safety and adverse events. Ten patients received CART19/20 cell products. One patient treated at DL1 (Patient 003) progressed prior to the end of DLT-monitoring period and was replaced per protocol and excluded from DLT evaluation. Infusions were generally well tolerated; 2 patients experienced a grade-1 infusion-related reaction (IRR) (Table 2). Grade-1 cytokine release syndrome (CRS) occurred in 6 patients; no grade 2 or higher CRS was observed in any patient. The median time from infusion to CRS was 8 days (range, 1-11) and the median duration was 2.5 days (range, 1-3). One dose of tocilizumab was given to Patient 009 for grade-1 CRS lasting greater than 48 hours. There were no cases of immune-effector cell associated neurotoxicity syndrome (ICANS), and no steroids were administered in the study for CRS or ICANS management. All patients experienced grade-3 or above adverse events consisting of generalized pain (n=2, 22%), hypotension (n=1, 11%), anemia (n=3, 33%), neutropenia (n=5, 56%), and thrombocytopenia (n=5, 56%). The only grade≥3 adverse events attributable to CART19/20 cells were anemia, thrombocytopenia, and neutropenia experienced by Patient 009.

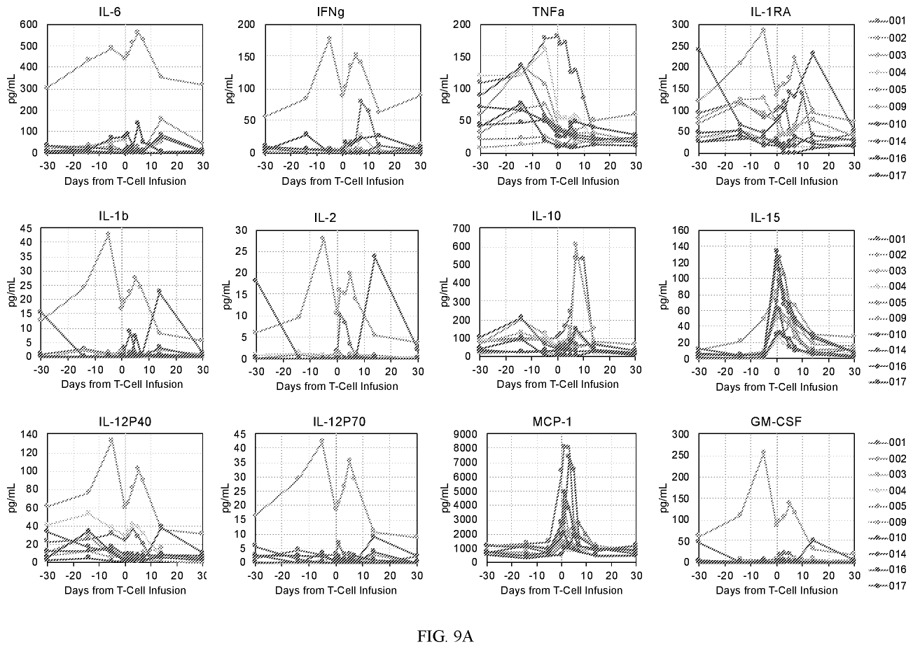

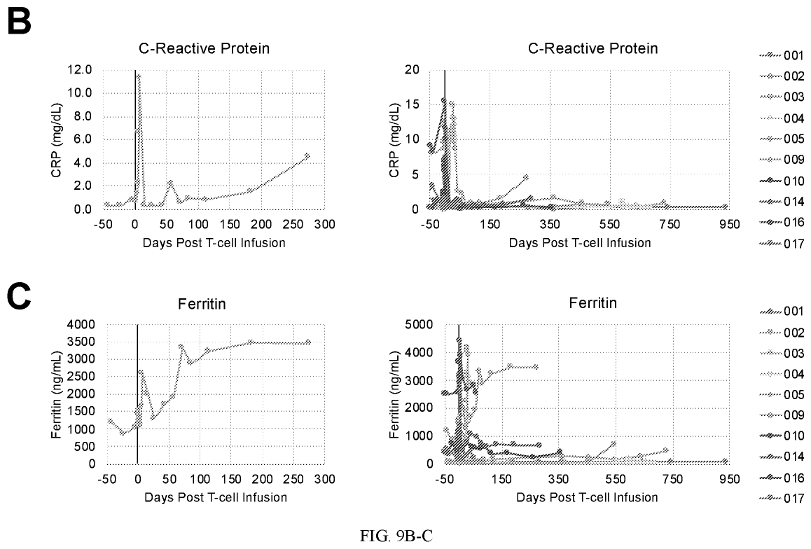

In contrast to the other grade 23 hematologic adverse events, the cytopenia in Patient 009 persisted beyond the expected recovery time from lymphodepletion chemotherapy and resulted in the only DLT observed in this study. Patient 009 had received autologous stem-cell transplant (ASCT) 11 months prior to receiving CART19/20 cell infusion, and exhibited elevated levels of multiple cytokines—including IL-6, IFN-γ, IL-IRA, IL-1b, IL-2, IL-12, and GM-CSF—prior to CART19/20 cell infusion, suggesting baseline inflammation and potentially contributing to post-infusion cytopenia (FIG. 9A). Post CART19/20 cell infusion, the patient experienced a typical, transient spike in C-reactive protein (CRP) and ferritin levels, but subsequently experienced a gradual increase in both that is unique among patients treated on this trial (FIGS. 9B and C). A bone marrow biopsy performed five months post CART19/20 cell infusion showed extensive, coalescing, non-necrotizing granulomatous inflammation. A trial of steroids was given with a transient improvement in pancytopenia. A repeat bone marrow biopsy performed ten months post CART19/20 cell infusion showed a hypocellular marrow with numerous histiocytes with increased hemophagocytic activity. Molecular characterization panel performed on this sample noted an expansion of the TET2 mutation to a variant allele frequency of 48% from a prior baseline of 1-3%, and a new ASXL1 mutation with a variant allele frequency of 65%. These findings suggest evolution of a myeloid neoplasm related to prior therapy.

Patient 009 elected to cease medical treatment shortly before the 12-month follow-up assessment. Given the severely hypocellular marrow and pancytopenia, T cells and peripheral mononuclear blood cells (PBMCs) could not be recovered in sufficient amounts to enable detailed follow-up analysis. Therefore, the patient's grade 5 hypocellular marrow is considered possibly related to CAR-T cells and possibly related to preconditioning chemotherapy. Biopsy taken shortly prior to the patient's election to end medical treatment showed no evidence of lymphoma.

No grade≥3 adverse events attributable to CART19/20 cell therapy has been observed in any other patient treated on this trial.

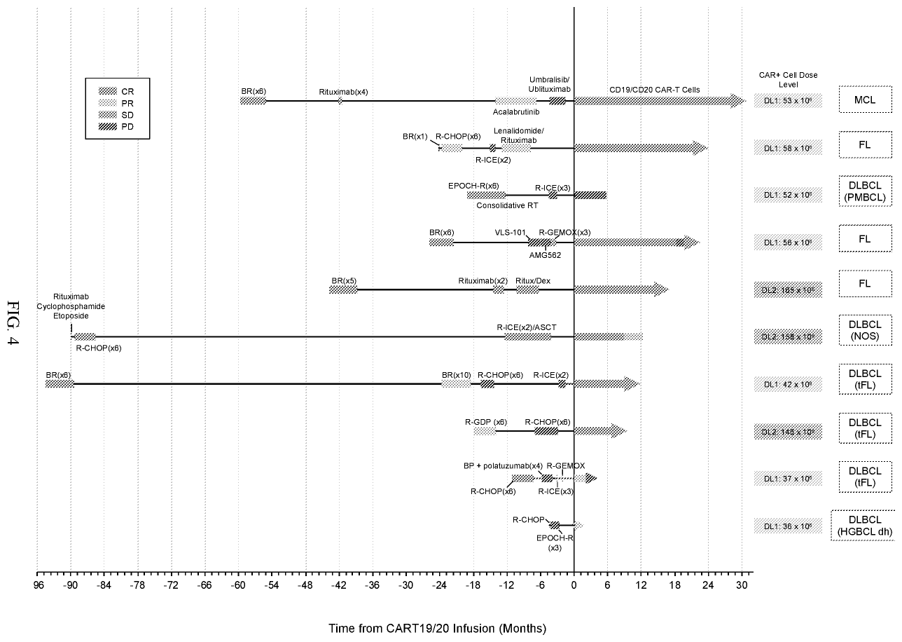

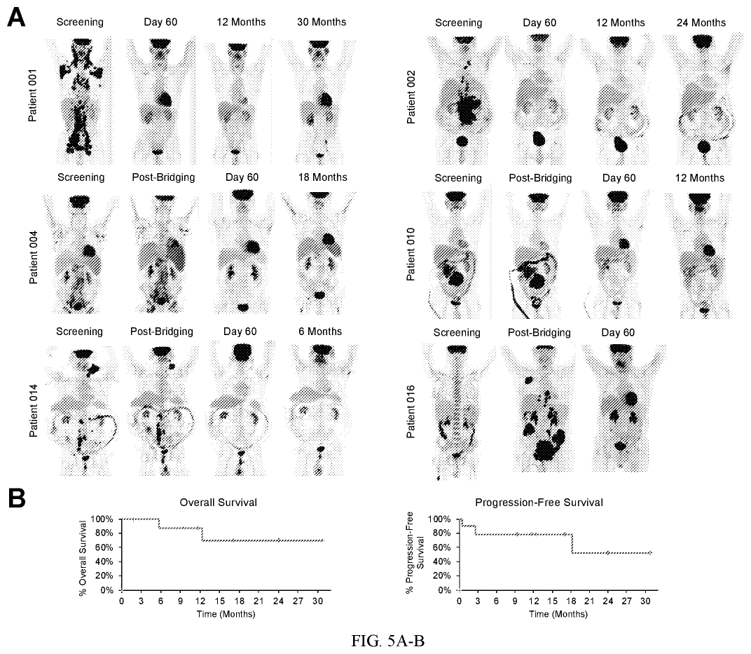

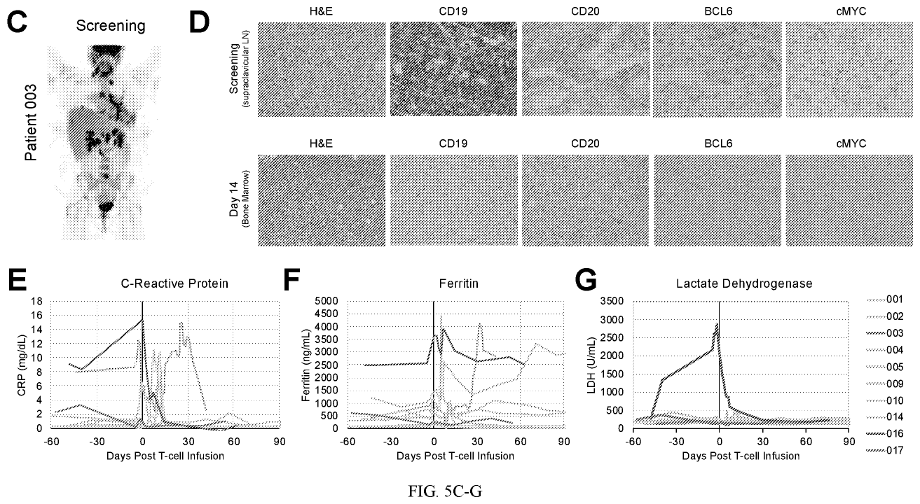

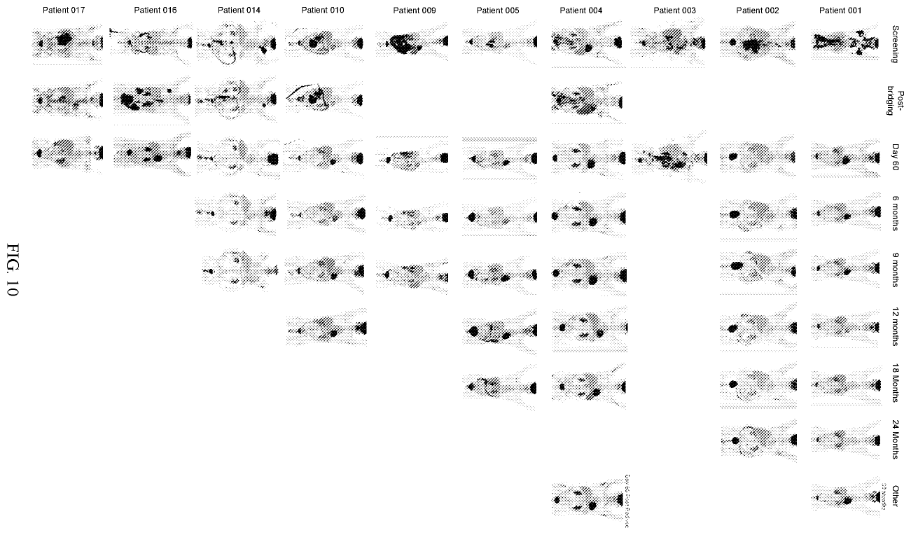

Response. Primary response assessment was performed 60 days after CART19/20 infusion by PET/CT scan. Nine out of ten patients responded to therapy (ORR=90%). Seven out of ten patients achieved a CR by the first disease assessment at day 60 (CR rate=70%), and two additional patients had a PR at day 60 (FIGS. 4 and 5A). Bridging therapy did not result in significant reduction of tumor burden in the majority of cases (FIG. 5A and FIG. 10), and there was no correlation between patient response and application of bridging therapy (Table 1). With a median follow-up of 17 months from time of CART19/20 cell infusion (range 2-30 months), the median overall survival (OS) and progression-free survival (PFS) were not reached (FIG. 5B).

The one patient who did not respond to therapy (Patient 003) was diagnosed with PMBCL (FIG. 5C), and had low ALC at the time of screening (0.17×10³ cells/μL). The patient exhibited marked clinical improvements, including reduced dyspnea and pain, in the week after CART19/20 cell infusion and was discharged from the hospital. However, disease progression was detected 14 days post CART19/20 infusion in a bone-marrow biopsy performed as part of routine evaluation for CAR-T cell infiltration and expansion. The screening biopsy of a supraclavicular lymph node from Patient 003 was positive for CD19, CD20, CD30 (patchy), BCL2, BCL6, and cMYC (FIG. 5D), with kappa light chain restriction; the screening biopsy was negative for CD10 and BCL1. In contrast, the bone-marrow sample obtained 14 days after CART19/20 infusion expressed CD30 and weak BCL2, and was negative for CD10, CD19, CD20, BCL1, BCL6 and cMYC (FIG. 5D), indicating a clonal shift in the tumor population.

To further understand the mechanism of dual antigen loss after CART19/20 treatment, an additional biopsy of the lung was obtained and analyzed by bulk RNA sequencing (RNA-seq). Consistent with the immunohistochemistry and flow cytometry results, RNA-seq showed loss of gene expression for CD19, CD20 and BCL6 (reads per kilobase transcript, per million mapped reads (RPKM)=0.12, 0.11, and 0.72, respectively), and low cMYC expression (RPKM=2.04). RNA-seq data further revealed a lack of CD22 expression (RPKM=0.07). Transcriptomic data interpretation is limited due to the lack of pre-treatment tissues for RNA-seq analysis. Nevertheless, the concomitant loss of BCL6 and cMYC—two antigens not under selective pressure from the CD19/20 bispecific CAR—within 14 days of CART19/20 cell infusion suggests the possibility of heterogenous tumor that contained a pre-existing antigen-negative subclone.

Patient 016, who was diagnosed with tFL, achieved a PR at day 60 (FIG. 5A) but progressed on day 90. Both Patients 003 and 016 exhibited elevated CRP prior to CART19/20 cell infusion (FIG. 5E), and both had elevated ferritin levels as of the last assessed time point (FIG. 5F). Following bridging therapy and prior to CART19/20 cell infusion, Patient 016 developed extremely high levels of lactate dehydrogenase (LDH) (FIG. 5G). The low ALC observed in Patient 003 and high pre-leukapheresis LDH observed in Patient 016 have both been shown to correlate with low odds for CR after CAR-T cell therapy (27). In contrast, Patient 017, who similarly achieved a PR at day 60, exhibited normal patterns of CRP, ferritin, and LDH levels (FIG. 5E-G), as well as a pre-leukapheresis ALC within normal range (Table 1). This patient was diagnosed with primary-refractory HGBCL with BCL6 and cMYC rearrangement, and remains in PR as of data cutoff.

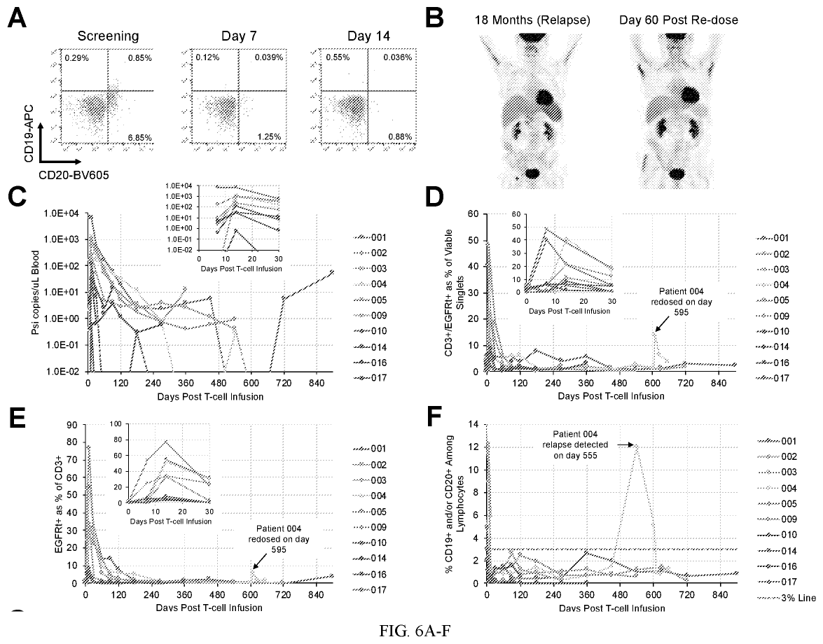

The seven patients who achieved a CR include one patient diagnosed with MCL, three patients with DLBCL (one de novo and two (FL) and three patients with FL (FIG. 4). All patients with FL were POD24, and the majority of patients were characterized by high tumor burdens (FIG. 10). Among the seven patients who achieved a CR, three (Patients 002, 009, and 014) had primary refractory disease, and a fourth (Patient 004) was refractory to anti-CD19 BiTE therapy (Table 1). In addition to anti-CD19 BiTE, Patient 004 was also refractory to ROR1-targeted antibody-drug conjugate therapy and progressed through the 2^{nd}-4^{th} lines of therapy within 5 months (FIG. 4). Flow cytometry analysis of Patient 004's peripheral blood at the time of screening indicated the presence of CD20⁺CD19^{dim/-} cells (FIG. 6A). This population was substantially reduced within 7 days of CART19/20 infusion, confirming CART19/20's ability to target tumor cells that have downregulated or lost CD19 expression. Patient 004 achieved a CR within 60 days and remained in CR until month 18, when reemergence of CD20⁺CD19⁺ FL was detected. Given the patient's history of therapy resistance and 18-month response to CART19/20, permission was obtained from the FDA to re-dose Patient 004 with 126×10⁶ CART19/20 cells, using extra aliquots of the same cell product as the first infusion. At day 60 post re-dosing, the patient again achieved a CR and remains in CR as of data cutoff (FIG. 6B).

CART19/20 cell persistence and B-cell aplasia. The presence of CART19/20 cells in peripheral blood post infusion were detected by both flow cytometry and droplet digital PCR (ddPCR). CAR copy number quantified by ddPCR consistently peaked at 14 days post infusion (FIG. 6C). At peak expansion, up to 48% of all viable singlets in PBMCs were CAR⁺ T cells (median 11.6%; range 1.5%-48.3%; FIG. 6D), and up to 77% of all CD3⁺ T cells were CAR⁺ (median 33.8%; range 3.7%-76.6%; FIG. 6E). At the time of data cutoff, all seven patients who were in PR (n=1) or CR (n=6) remained in B-cell aplasia based on flow cytometry analysis of peripheral blood (FIG. 6F), indicating functional persistence of CART19/20 cells.

For Patient 004, who remained in CR for 18 months before relapsing. CAR+ T cells dropped below ddPCR detection at 12 months but reemerged at the time of relapse (FIG. 6C), suggesting the presence of a residual CART19/20 cell population that expanded upon tumor relapse but was insufficient to prevent tumor outgrowth. The patient experienced a second wave of CAR+ T-cell expansion after receiving the second dose of CART19/20 cells (FIG. 6D,E). The more modest expansion post re-dosing compared to the initial peak suggests the possibility of resistance development toward the CAR-T cell product, but the re-dose was nonetheless successful in re-establishing a CR for this patient. The lower tumor burden and thus lower antigen load at relapse compared to initial screening (FIGS. 5A and 3B) could also have contributed to the lower CAR-T cell expansion upon re-dose. B cells were undetectable in the peripheral blood of Patient 004 after the first CART19/20 cell infusion, was detected at the time of relapse, and dropped below detection again after receiving the second dose of CART19/20 cells, consistent with response to therapy (FIG. 5F).

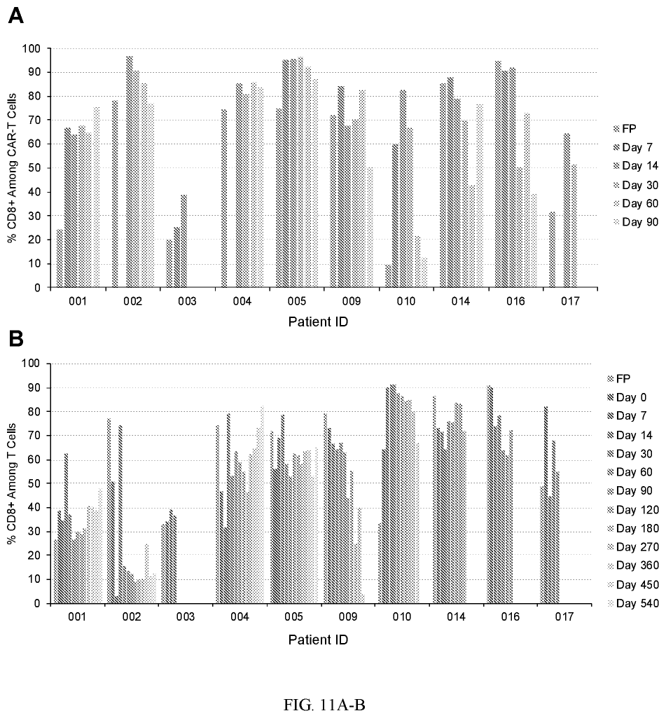

As previously noted, CART19/20 cell products were either CD4- or CD8-dominant at the time of cryopreservation, depending on whether CD14/CD25 depletion was performed (FIG. 3E). Interestingly, regardless of the CD4: CD8 ratio in the cryopreserved cell product, Both CAR-expressing T cells and total T cells rapidly became CD8-dominant after CART19/20 cell infusion in all but one patient (FIG. 11). The CD8 dominance declines over time, mirroring recently reported findings in a decade-long follow up of two patients with leukemia treated with CD19 CAR-T cell therapy (28).

Cytokine assessments. Consistent with clinical presentation of grade 1 CRS for over 48 hours. Patient 009 showed elevated levels of most cytokines in peripheral blood compared to other patients in the trial (FIG. 9A). As a whole, cytokine levels observed in patients treated with CART19/20 cells are similar to or substantially lower than values reported in earlier trials for single-input CD19 CAR-T cell therapies (3,5,29-31) and CD19/CD20 bispecific CAR-T cell therapies (18,32). The relatively low peak cytokine levels in patients treated with CART19/20 may be a contributing factor to the strong safety profile observed in this trial to date. Taken together, results of this phase-1 clinical trial indicate CART19/20 cell therapy is safe and effective for the treatment of NHL (Table 3).

### B. Discussion

CD19 CAR-T cell therapy became the first FDA-approved gene-modified cell therapy in 2017, and is making rapid progress toward incorporation in earlier lines of treatment for B-cell malignancies. However, antigen escape and lack of T-cell expansion and persistence remain key factors that limit the frequency as well as durability of response in patients treated with CD19 CAR-T cell therapy (1-5,15-17,33,34). Early results of the inventors' phase-1 trial on CART19/20 cell therapy demonstrate that dual-targeting of CD19 and CD20 using a molecularly optimized single-chain bispecific CAR (6,7) is safe and effective in patients with relapsed/refractory NHL, with potentially higher CR rates and lower toxicity using lower T-cell doses compared to previously reported clinical candidates (18,35).

In this trial, ten patients were treated with a median dose of 55×10⁶ CART19/20 cells, with no patient receiving more than 165×10⁶ cells. These dosing levels are substantially lower than the median dosage level used in clinical trials evaluating other CD19/CD20 bispecific CAR clinical candidates (18,35) as well as in pivotal trials for single-input CD19 CAR-T cell products including axicabtagene ciloleucel (36,37), tisagenlecleucel (38,39), and lisocabtagene maraleucel (40). Despite the low dosage used, strong efficacy was observed in this trial (90% ORR; 70% CR rate), with a heavily pretreated patient population carrying high tumor burden and highly aggressive disease. To date, all but one patient who achieved a CR has remained in CR. The only patient to experience a relapse did so after 18 months, and the patient subsequently returned to CR after receiving a second dose of CART19/20 cells. With a median follow-up of 17 months, median PFS has not been reached in this trial.

Importantly, the strong efficacy observed in this trial was achieved with no ICANS of any grade and no CRS above grade 1 after CART19/20 infusion. To date, the vast majority of grade≥3 adverse events experienced by patients treated with CART19/20 were toxicities attributable to bridging and lymphodepletion chemotherapies in patients with substantial previous chemotherapy exposure. One patient death (Patient 009) from grade 5 hypocellular marrow is considered possibly related to treatment, and possibly related to myeloid neoplasm consequent to therapies received prior to CART19/20. Although no evidence of lymphoma was detected in the patient at the time of elected transition to comfort care, this case underscores the potential benefit of providing CAR-T cell therapy as an earlier line of treatment, which could reduce the total amount of chemotherapy exposure and related toxicities to the patient. Overall, the safety profile observed in this trial compares favorably with prior reports of CD19-targeted as well as CD19/CD20-targeted CAR-T cell therapies (18,35-41), and supports the possibility of combining strong efficiency with a high level of safety.

The trial reported here included one patient with MCL, three patients with FL, and five patients with DLBCL of various subtypes. It is plausible that different DLBCL subtypes have different response rates to CAR-T cell therapy, although no trial reported to date has been statistically powered to evaluate the responses of different DLBCL subtypes to CD19 CAR-T cell therapy (36,39,40). FL is often described as an indolent disease, and has shown favorable response rates to therapy (38). It should be noted that in this trial, all three patients with FL were POD24, including one patient who was primary refractory (Patient 002), and another patient who was refractory to the three lines of therapy immediately before CART19/20, including CD19-targeted BiTE (Patient 004). Therefore, the subjects enrolled in this trial represented a particularly challenging subset of patients with FL, and all achieved a CR post CART19/20 cell infusion. In addition to four other patients with MCL or DLBCL who also achieved a CR, one patient with primary refractory, double-hit HGBCL (Patient 017) achieved a PR at day 60. This patient will continue to be monitored for the possibility of a delayed CR. Overall, CART19/20 cell therapy has shown robust efficacy in a highly pretreated patient population with challenging disease profiles.

The only patient who did not respond to CART19/20 therapy to date (Patient 003) had PMBCL refractory to R-ICE salvage chemotherapy. Despite notable clinical improvement in the week post CART19/20 infusion, this patient experienced a rapid emergence of CD19⁻CD20⁻ tumor cells that had also lost BCL6 and cMYC expression within 14 days of CART19/20 treatment. The number of protein-expression changes combined with the rapidity of clonal shift suggests a pre-existing tumor subpopulation that was able to swiftly expand after CART19/20 cells eliminated the originally dominant CD19⁺CD20⁺ tumor cells. The emergence of CD19⁻ tumor was previously reported in the relapse of a patient with PMBCL treated with CD19 CAR-T cell therapy, and sequencing analysis indicated the likely cause was a pre-existing clone that expanded under CD19-targeted selective pressure (42).

Promoting T-cell persistence and function through the generation of cell products enriched in naïve and memory cell types was a key element of the CART19/20 therapy design. CART19/20 products were generated from a T_{N/MEM} cell population obtained through bead-based enrichment of CD62L expression, and the final products retained substantial central-memory T-cell content. Contrary to original expectations, the presence of the CD14⁺ cells did not adversely impact the inventors' ability to successfully manufacture CART19/20 cell products with high T-cell purity and clinical efficacy. The only clearly measurable impact of CD14⁺ cell presence was the CD4:CD8 ratio of the final CART19/20 product, with the presence of CD14⁺ cells leading to CD4-dominant products while the depletion of CD14⁺ cells from the starting material led to CD8-dominant products. However, based on the results to date, there is no correlation between the CD4:CD8 ratio and clinical outcome. Similarly, the lack of Treg depletion through CD25 did not show measurable impact on treatment outcome, which is consistent with prior reports (43).

A key question of interest is how the CART19/20 cells evaluated in this trial are able to achieve the high level of efficacy at low dosage level and without incurring the type of toxicities observed in comparable trials. As previously reported, the CD19/CD20 bispecific CAR used here had been optimized at a sequence level to maximize efficacy (6). Safety was not a consideration in the CAR engineering process. However, the robust efficacy enabled the use of a very low cell dose to achieve therapeutic benefit, and the low cell dose may in turn have contributed to the favorable safety profile observed in this trial. Unlike conventional CD19 CAR-T cell therapy, CART19/20 cells are capable of eliminating CD19^{dim/-} tumor cells within 7-14 days of T-cell infusion (FIG. 6A). In addition to safeguarding against antigen escape, the rapidity with which CART19/20 eliminates tumor cells may contribute to the limited toxicity observed—i.e., the bulk of the tumor could be eliminated before T cells reach peak expansion at 14 days post infusion, thus providing temporal separation between high tumor burden and large CAR-T cell numbers in the patient. Finally, the use of T_{N/MEM}-derived cells may further contribute to the potency and safety profile of CART19/20 cells by reducing peak cytokine levels while retaining long-term anti-tumor efficacy.

In summary, early results from this phase-1 trial indicate that autologous, CD19/CD20 dual-targeting CAR-T cells enriched in naïve and memory phenotypes are safe and highly efficacious in the treatment of relapsed/refractory NHL. These results suggest potent clinical efficacy can be achieved while avoiding severe toxicities typically associated with CAR-T cell therapy, and highlight the utility of dual-antigen targeting cell-based immunotherapy.

### C. Methods

Trial Design. A prospective, first-in-human phase I clinical trial assessing CART19/20 in adult patients with relapsed/refractory NHL and CLL/SLL was initiated at the University of California, Los Angeles (UCLA) Medical Center. The study was approved by the UCLA Institutional Review Board (IRB) and registered with ClinicalTrials.gov (NCT04007029). Informed written consent was obtained in accordance with the Declaration of Helsinki, International Conference on Harmonization (ICH) Good Clinical Practice (GCP), US Code of Federal Regulations for Protection of Human Subjects, the Health Insurance Portability and Accountability Act, and local regulations. Data monitoring was conducted by the UCLA Jonsson Comprehensive Cancer Center (JCCC) Data Safety and Monitoring Board (DSMB). The primary endpoint was safety, defined by the incidence and severity of dose-limiting toxicities (DLTs), as well as determination of the maximum tolerated dose (MTD). Secondary endpoints included clinical efficacy measures, and analysis of CAR T-cell persistence and B-cell aplasia. Exploratory endpoint was cytokine release syndrome analysis.

Patient enrollment and eligibility. Patients eligible for the clinical trial were ≥18 years old with diffuse large B-cell lymphoma (DLBCL) or primary mediastinal B-cell lymphoma (PMBCL) after ≥2 prior lines of therapy, or with mantle-cell lymphoma (MCL), follicular lymphoma (FL), chronic lymphocytic leukemia (CLL) or small lymphocytic lymphoma (SLL) after ≥3 prior lines of therapy. Transformed indolent lymphomas, including Richter transformation, were eligible and previous lines of therapy were considered from the time of transformation. Autologous stem cell transplant (ASCT) recipients were allowed in the study. Patients were required to have greater than 30% positivity in malignant cells of CD19 and/or CD20, as well as measurable tumor burden on PET/CT. Any NHL- or CLL/SLL-directed therapy, including corticosteroids, within 14 days of initiation of lymphodepletion chemotherapy was exclusionary. After leukapheresis, bridging therapy was permitted at the investigator's discretion. Lymphodepletion chemotherapy, consisting of fludarabine 30 mg/m² and cyclophosphamide 500 mg/m², was administered on day-5 through day-3 before infusion.

Toxicity assessment. Adverse events were recorded for all treated patients until disease relapse or death, with incidence and severity graded using the Common Terminology Criteria for Adverse Events (CTCAE) version 5.0. CRS was graded according to the ASTCT and Lee criteria, with the former guiding treatment (44), For neurotoxic events, the ASTCT criteria was for scoring and treatment, with specific guidance to key disorders outlined by Neelapu et al. (45).

Response assessment. The clinical response in lymphoma was evaluated with the criteria defined by The Revised Cheson Response Criteria and Lugano Classification (46,47). The overall response rate (ORR) was defined as the total of complete responses (CR) and partial responses (PR).

CART19/20 cell manufacturing. Fresh patient leukapheresis products were analyzed by flow cytometry to determine the CD3⁺, CD62L⁺, CD14⁺, and CD25⁺ cell frequency. When needed, cells were labeled with anti-CD14 and anti-CD25 CliniMACS microbeads and depleted using the CliniMACS Plus system (Miltenyi Biotec). Remaining cells were subsequently enriched for CD62L using the same system to yield T_{N/MEM} cells. T_{N/MEM} cells were activated with TransAct (Miltenyi Biotec) and transduced with GMP-grade lentivirus. Patient cells were expanded ex vivo for a total of 12-16 days prior to cryopreservation.

Flow cytometry analysis of lymphocytes. For pre- and post-isolation leukapheresis product analysis, samples were stained with antibodies for CD3, CD14, CD25, and CD62L. For CART19/20 final product analysis, cryopreserved products were thawed, washed with PBS, and stained with antibodies for CD3 and epidermal growth factor receptor (EGFR). The CD19/CD20 bispecific CAR is co-expressed with a truncated, non-signaling EGFR (EGFRt), thus EGFRt serves as a proxy for CAR expression. For patient peripheral blood analysis, blood samples were collected in ethylenediaminetetraacetic acid (EDTA) tubes, and peripheral blood mononuclear cells (PBMCs) were collected using the SepMate system (STEMCELL Technologies) following manufacturer's protocol. Isolated PBMCs were frozen until use. Thawed cells were surface stained with antibody panels for T-cell phenotype (CD3, CD4, CD8, CD62L, CD45RA, CD45RO, and EGFR), or B-cell quantification (CD19, CD20, CD56, CD3, CD14, and SYTOX Blue). Flow cytometry was performed on an Attune N×T flow cytometer (ThermoFisher), and data were analyzed using FlowJo v.10.7.1 (FlowJo, LLC). Gating strategies are shown in FIG. 10.

Cytokine analysis. Patient peripheral blood was collected into red-top tubes containing no anti-coagulant or preservative, allowed to clot for 30 minutes in the upright position at room temperature, transferred to a conical tube, and centrifuged at 900×g for 10 minutes. The supernatant was frozen in aliquots until use. Cytokine analysis was performed by the UCLA Immune Assessment Core Facility using the Luminex 38-plex human cytokine chemokine panel, following manufacturer's protocols.

Statistical analysis. Descriptive statistics were measured by median and range for continuous variables and counts and percentages for categorical variables. Patients were censored at the time of the last follow up. Duration of remission (DOR), progression-free survival (PFS), and overall survival (OS) were estimated by Kaplan-Meier method. The statistical software packages used was IBM SPSS statistics. DOR was defined as the time of the first documented CR/PR until the first date that recurrent or progressive disease is objectively documented, or until death. PFS was defined as the time of CART19/20 infusion until documentation of objective disease progression or death due to any cause. OS was measured from the date of CART19/20 infusion in the clinical trial until death.

### D. Tables

TABLE 1
Patient demographics and treatment history

| | | | Prior Therapies | | | |
|---|---|---|---|---|---|---|
| Patient | | | # of | | | Disease |
| ID | Age/Sex | Diagnosis | Lines | Therapies | Notes | Stage |
| 001 | 60/M | MCL | 4 | Bendamustine/rituximab | | IV |
| | | | | Rituximab | | |
| | | | | Acalabrutinib | | |
| | | | | Umbralisib/ublituximab | | |
| 002 | 58/M | FL | 4 | Bendamustine/rituximab | Primary | IV |
| | | | | R-CHOP | Refractory | |
| | | | | R-ICE | | |
| | | | | Lenalidomide/rituximab | | |
| 003 | 29/F | DLBCL | 2 | EPOCH-R | | IV |
| | | (PMBGL) | | R-ICE | | |
| 004 | 62/M | FL | 4 | Bendamustine/rituximab | POD24; | IV |
| | | | | ROR1-Targeting Antibody-Drug | Refractory to | |
| | | | | Conjugate | CD19 BiTE | |
| | | | | Anti-CD19 BiTE | | |
| | | | | Rituximab/gemcitabine/ | | |
| | | | | oxaliplatin | | |
| 005 | 35/F | FL | 3 | Bendamustine rituximab | POD24 | IV |
| | | | | Rituximab | | |
| | | | | Rituximab/dexamethasone | | |
| 009 | 67/M | DLBCL | 4 | Rituximab/cyclophosphamide/ | Primary | IV |
| | | (NOS) | | etoposide | Refractory | |
| | | | | R-CHOP | | |
| | | | | R-ICE | | |
| | | | | ASCT | | |
| 010 | 56/M | DLBCL | 3 | Bendamustine/rituximab | | IV |
| | | (tFL) | | R-CHOP | | |
| | | | | R-ICE | | |
| 014 | 64/M | DLBCL | 2 | Rituximab/gemcitabine/ | Primary | IV |
| | | (tFL) | | dexamethasone/carboplatin | Refractory | |
| | | | | R-CHOP | | |
| 016 | 70/M | DLBCL | 4 | R-CHOP | | IV |
| | | | | BP + polatuzumab | | |
| | | (tFL) | | R-ICE | | |
| | | | | R-GEMOX | | |
| 017 | 39/M | DLBCL | 2 | R-CHOP | Primary | IV |
| | | (HGBCL | | EPOCH-R | Refractory | |
| | | double-hit) | | | | |

| | ALC at | | | CAR + | | | Best |
|---|---|---|---|---|---|---|---|
| Patient | Screening | | Dosing | T Cells | CRS, | ICANS | Response |
| ID | (×10\hat{ }3/μL) | Bridging Therapy | Level | Received | (Grade) | (Grade) | (PFS) |
| 001 | 0.40 | Cyclophosphamide | DL1 | 53 × 10\hat{ }6 | Yes (1) | No | CR (30+) |
| 002 | 0.96 | Cyclophosphamide | DL1 | 58 × 10\hat{ }6 | Yes (1) | No | CR (24+) |
| | | EPOCH-R | | | | | |
| 003 | 0.17 | R-GEMOX | | 52 × 10\hat{ }6 | Yes (1) | No | PD |
| 004 | 1.02 | R-GEMOX | DL1 | 56 × 10\hat{ }6 | Yes (1) | No | CR (18) |
| | | (did not | | | | | |
| | | tolerate/complete | | | | | |
| | | rituximab) | | | | | |
| 005 | 1.12 | None | DL2 | 165 × 10\hat{ }6 | Yes (1) | No | CR (18+) |
| 009 | 2.09 | GEMOX | DL2 | 158 × 10\hat{ }6 | Yes (1) | No | CR (9+)* |
| 010 | 0.63 | ICE | DL1 | 42 × 10\hat{ }6 | No | No | CR (12+) |
| 014 | 2.60 | ICE | DL2 | 146 × 10\hat{ }6 | No | No | CR (9+) |
| 016 | 1.20 | GEMOX + | DL1 | 37 × 10\hat{ }6 | Yes (1) | No | PR (2) |
| | | R-GEMOX | | | | | |
| 017 | 1.88 | ICE | DL1 | 36 × 10\hat{ }6 | No | No | PR (2+) |

*Patient 009 elected to transition to comfort care shortly prior to the scheduled 12-month follow-up. Biopsy performed at that time showed no evidence of lymphoma. However, CR at 12 month was not verified with PET scan.
Abbreviations:
ALC: absolute lymphocyte count;
CRS: cytokine release syndrome;
ICANS: immune-effector cell associated neurotoxicity syndrome;
PFS: progression-free survival;
MCL, mantle-cell lymphoma;
FL, follicular lymphoma;
PMBCL, primary mediastinal B-cell lymphoma;
DLBCL, diffuse large B-cell lymphoma;
NOS, no other specification;
tFL, transformed follicular lymphoma;
HGBCL: high-grade B-cell lymphoma;
R-CHOP: rituximab/cyclophosphamide/doxorubicin/hydrochloride/vincristine sulfate/prednisone;
R-ICE: rituximab/ifosfamide/carboplatin/etoposide;
EPOCH-R: etoposide/prednisone/vincristine/cyclophosphamide/doxorubicin/rituximab;
ASCT: autologous stem-cell transplate;
R-GEMOX: Rituximab/gemcitabine/oxaliplatin;
ICE: ifosfamide/carboplatin/etoposide;
CR: complete response;
PR: partial response;
PD: progressive disease

TABLE 2
Adverse events

| TEAE | All Grades (%) | Grade 3+ (%) |
|---|---|---|
| Any | 9 (100%) | 9 (100%) |
| IRR | 2 (22%) | 0 |
| CRS | 6 (66%) | 0 |
| ICANS | 0 | 0 |
| Infection | 0 | 0 |
| Pain | 4 (44%) | 2 (22%) |
| Fatigue | 8 (89%) | 0 |
| Hypotension | 2 (22%) | 1 (11%) |
| Headache | 2 (22%) | 0 |
| ALT increased | 1 (13%) | 0 |
| AST increased | 1 (13%) | 0 |
| Anemia | 9 (100%) | 3 (33%) |
| Neutropenia | 8 (89%) | 5 (56%) |
| Thrombocytopenia | 8 (89%) | 5 (56%) |
| Nausea | 6 (67%) | 0 |
| Vomiting | 2 (22%) | 0 |
| Diarrhea | 2 (2%) | 0 |

TABLE 3
Trial Comparisons

| CAR | | | | |
|---|---|---|---|---|
| Type | Trial | ORR | CR Rate | Median PFS |
| CD19/ | UCLA | 90% | Overall 70% ( 7/10) | Not Reached |
| CD20/CAR | | ( 9/10) | 36M-58M: 57% ( 4/7; 1 (25%) relapse) | (median |
| | | | 146M-165M, 100% ( 3/3) | follow up = |
| | | | | 17 months) |
| CD19/ | Medical | 82% | Overall: 64% ( 14/22); 29% ( 2/7) | 9.1 months |
| CD20/CAR | College of | ( 18/22) | with cryopreserved products | |
| | Wisconsin | | 17.5M: 33% (⅓) | |
| | (Lentigen) | | 52.5M: 33% (⅓); 1 (100%) relapse) | |
| | | | 175M: 75% ( 12/16; 4 (33%) relapse) | |
| CD19/ | Chinese PLA | 80% | Overall: 70% ( 61/87) | 27.6 months |
| CD20/CAR | General | ( 70/87) | 39M-55M: 100% ( 2/2) | |
| | Hospital | | 126M-196M: 61% ( 11/18; 4 (36%) | |
| | | | relapse) | |
| | | | 203M-294M: 68% ( 17/25; 7 (41%) | |
| | | | relapse) | |
| | | | 308M-378M: 69% ( 9/13; 3 (33%) | |
| | | | relapse) | |
| | | | 413M-560M: 76% ( 22/29; 4 (18%) | |
| | | | relapse) | |
| CD19 | ZUMA1 | 83% | Overall: 58% ( 59/101) | 5.9 months |
| CAR | | ( 84/101) | Target dose: 2M/kg = 140M for | |
| | | | 70-kg patient | |
| CD19 | JULIET | 53% | Overall: 39% ( 45/115) | 2.9 months |
| CAR | | ( 61/115) | Target dose: 500M | |
| CD19 | TRANSCEND | 73% | Overall: 53% ( 136/256) | 6.8 months |
| CAR | NHL 001 | ( 186/256) | 50M: 60% ( 24/40) | |
| | | | 100M: 52% ( 88/169) | |
| | | | 150M: 51% ( 21/41) | |

| CAR | | | | |
|---|---|---|---|---|
| Type | CRS | ICANS | Notes on Cell | Reference |
| CD19/ | 66% overall | None | All products | This Study |
| CD20/CAR | all grade 1 | | cryopreserved; 11 | |
| | | | patients underwent | |
| | | | leukapheresis and | |
| | | | 10 dosed (91%) | |
| CD19/ | 64% overall | 32% overall | 7 patients dosed | Shah et al., |
| CD20/CAR | 5% grade 3+ | 14% grade 3+ | with cryopreserved | Nat. Med., |
| | | | products; 15 patients | 2020 |
| | | | dosed with fresh | |
| | | | products; 26 patients | |
| | | | underwent leukapheresis | |
| | | | and 22 dosed (85%) | |
| CD19/ | 70% overall | 17% overall | All products | Zhang et al., |
| CD20/CAR | 10% grade 3+ | 2% grade 3 | cryopreserved; 92 | Leukemia, |
| | | | patients underwent | 2021 |
| | | | leukaperesis and 87 | |
| | | | dosed (95%) | |
| CD19 | 99% overall | 67% overall | All products | Locke |
| CAR | 11% grade 3+ | 32% grade 3+ | cryopreserved; 119 | et al., |
| | | | patients enrolled | Lancet, |
| | | | and 108 dosed (91%) | 2018 |
| CD19 | 57% overall | 20% overall | All products | Schuster |
| CAR | 25% grade 3+ | 11% grade 3+ | cryopreserved; 167 | et al., |
| | | | patients enrolled | Lancet, |
| | | | and 115 dosed (69%) | 2021 |
| CD19 | 42% overall | 30% overall | All products | Abramson |
| CAR | 2% grade 3+ | 10% grade 3+ | cryopreserved; 344 | et al., |
| | | | patients underwent | Lancet, |
| | | | leukapheresis and 294 | 2020 |
| | | | dosed (85%) | |

Dosage levels for trials where dosage level was reported as per kg, values are shown in the table assuming 70 kg per patient.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The references cited in the disclosure, to the extent that they provide exemplary procedural or other details supplementary to those set forth herein, are specifically incorporated herein by reference.

The references cited herein (including the following references), to the extent that they provide exemplary procedural or other details supplementary to those set forth herein, are specifically incorporated herein by reference.
1. Maude S L, Frey N. Shaw P A, Aplenc R, Barrett D M, Bunin N J, et al. Chimeric antigen receptor T cells for sustained remissions in leukemia. N Engl J Med 2014; 371 (16): 1507-17 doi 10.1056/NEJMoa1407222.
2. Neelapu S S, Locke F L, Bartlett N L, Lekakis L J, Miklos D B, Jacobson C A, et al. Axicabtagene Ciloleucel CAR T-Cell Therapy in Refractory Large B-Cell Lymphoma. N Engl J Med 2017; 377 (26): 2531-44 doi 10.1056/NEJMoa1707447.
3. Lee D W, Kochenderfer J N, Stetler-Stevenson M, Cui Y K, Delbrook C. Feldman S A, et al. T cells expressing CD19 chimeric antigen receptors for acute lymphoblastic leukaemia in children and young adults: a phase 1 dose-escalation trial. Lancet 2015; 385 (9967): 517-28 doi 10.1016/S0140-6736 (14) 61403-3.
4. Gardner R A, Finney O, Annesley C, Brakke H, Summers C, Leger K, et al. Intent-to-treat leukemia remission by CD19 CAR T cells of defined formulation and dose in children and young adults. Blood 2017; 129 (25): 3322-31 doi 10.1182/blood-2017-02-769208.
5. Maude S L, Laetsch T W, Buechner J, Rives S. Boyer M, Bittencourt H, et al. Tisagenlecleucel in Children and Young Adults with B-Cell Lymphoblastic Leukemia. N Engl J Med 2018; 378 (5): 439-48 doi 10.1056/NEJMoa1709866.
6. Zah E, Lin M Y. Silva-Benedict A, Jensen M C, Chen Y Y. T Cells Expressing CD19/CD20 Bispecific Chimeric Antigen Receptors Prevent Antigen Escape by Malignant B Cells. Cancer Immunol Res 2016; 4 (6): 498-508 doi 10.1158/2326-6066.CIR-15-0231.
7. Zah E, Lin M Y, Silva-Benedict A, Jensen M C. Chen Y Y. ADDENDUM: T Cells Expressing CD19/CD20 Bispecific Chimeric Antigen Receptors Prevent Antigen Escape by Malignant B Cells. Cancer Immunol Res 2016; 4 (7): 639-41 doi 10.1158/2326-6066.CIR-16-0108.
8. Salles G. Barrett M, Foa R, Maurer J, O'Brien S, Valente N, et al. Rituximab in B-Cell Hematologic Malignancies: A Review of 20 Years of Clinical Experience. Adv Ther 2017; 34 (10): 2232-73 doi 10.1007/s12325-017-0612-x.
9. Johnson N A, Leach S, Woolcock B, deLeeuw R J, Bashashati A, Sehn L H, et al. CD20 mutations involving the rituximab epitope are rare in diffuse large B-cell lymphomas and are not a significant cause of R-CHOP failure. Haematologica 2009; 94 (3): 423-7 doi 10.3324/haematol.2008.001024.
10. Till B G, Jensen M C, Wang J, Chen E Y, Wood B L, Greisman H A, et al. Adoptive immunotherapy for indolent non-Hodgkin lymphoma and mantle cell lymphoma using genetically modified autologous CD20-specific T cells. Blood 2008; 112 (6): 2261-71 doi 10.1182/blood-2007-12-128843.
11. Till B G, Jensen M C. Wang J, Qian X. Gopal A K. Maloney D G, et al. CD20-specific adoptive immunotherapy for lymphoma using a chimeric antigen receptor with both CD28 and 4-1B B domains: pilot clinical trial results. Blood 2012; 119 (17): 3940-50 doi 10.1182/blood-2011-10-387969.
12. Wang Y. Zhang W Y, Han Q W. Liu Y. Dai H R, Guo Y L, et al. Effective response and delayed toxicities of refractory advanced diffuse large B-cell lymphoma treated by CD20-directed chimeric antigen receptor-modified T cells. Clin Immunol 2014; 155 (2): 160-75 doi 10.1016/j.clim.2014.10.002.
13. Zhang W Y. Wang Y, Guo Y L, Dai H R, Yang Q M, Zhang Y J, et al. Treatment of CD20-directed Chimeric Antigen Receptor-modified T cells in patients with relapsed or refractory B-cell non-Hodgkin lymphoma: an early phase IIa trial report. Signal Transduct Target Ther 2016; 1:16002 doi 10.1038/sigtrans.2016.2.
14. Wudhikarn K. Palomba M L, Pennisi M, Garcia-Recio M, Flynn J R, Devlin S M, et al. Infection during the first year in patients treated with CD19 CAR T cells for diffuse large B cell lymphoma. Blood Cancer J 2020; 10 (8): 79 doi 10.1038/s41408-020-00346-7.
15. Hsich E M, Scherer L D, Rouce R H. Replacing CAR-T cell resistance with persistence by changing a single residue. J Clin Invest 2020; 130 (6): 2806-8 doi 10.1172/JCI136872.
16. Fraietta J A, Lacey S F, Orlando E J, Prutcanu-Malinici I, Gohil M, Lundh S, et al. Determinants of response and resistance to CD19 chimeric antigen receptor (CAR) T cell therapy of chronic lymphocytic leukemia. Nat Med 2018; 24 (5): 563-71 doi 10.1038/s41591-018-0010-1.
17. Turtle C J, Hanafi L A, Berger C, Gooley T A, Cherian S. Hudecek M, et al. CD19 CAR-T cells of defined CD4+: CD8+ composition in adult B cell ALL patients. J Clin Invest 2016; 126 (6): 2123-38 doi 10.1172/JCI85309.
18. Shah N N, Johnson B D, Schneider D, Zhu F. Szabo A, Keever-Taylor C A, et al. Bispecific anti-CD20, anti-CD19 CAR T cells for relapsed B cell malignancies: a phase 1 dose escalation and expansion trial. Nat Med 2020; 26 (10): 1569-75 doi 10.1038/s41591-020-1081-3.
19. Berger C. Jensen M C, Lansdorp P M, Gough M, Elliott C, Riddell S R. Adoptive transfer of effector CD8+ T cells derived from central memory cells establishes persistent T cell memory in primates. J Clin Invest 2008; 118 (1): 294-305 doi 10.1172/JCI32103.
20 Xu Y. Zhang M, Ramos C A, Durett A, Liu E, Dakhova O, et al. Closely related T-memory stem cells correlate with in vivo expansion of CAR.CD19-T cells and are preserved by IL-7 and IL-15. Blood 2014; 123 (24): 3750-9 doi 10.1182/blood-2014-01-552174.
21. Zah E, Nam E, Bhuvan V. Tran U, Ji B Y, Gosliner S B, et al. Systematically optimized BCMA/CS1 bispecific CAR-T cells robustly control heterogeneous multiple myeloma. Nat Commun 2020; 11 (1): 2283 doi 10.1038/s41467-020-16160-5.
22. Yam P Y, Li S, Wu J, Hu J, Zaia J A, Yec J K. Design of HIV vectors for efficient gene delivery into human hematopoictic cells. Mol Ther 2002; 5 (4): 479-84 doi 10.1006/mthe.2002.0558
23. Wang X, Borquez-Ojeda O. Stefanski J, Du F, Qu J, Chaudhari J, et al. Depletion of high-content CD14 (+) cells from apheresis products is critical for successful transduction and expansion of CAR T cells during large-scale cGMP manufacturing. Mol Ther Methods Clin Dev 2021; 22:377-87 doi 10.1016/j.omtm.2021.06.014.
24. Colombo M P, Piconese S. Regulatory-T-cell inhibition versus depletion: the right choice in cancer immunotherapy. Nat Rev Cancer 2007; 7 (11): 880-7 doi 10.1038/nrc2250.
25. Kunkele A, Brown C, Beebe A, Mgebroff S, Johnson A J, Taraseviciute A, et al. Manufacture of Chimeric Antigen Receptor T Cells from Mobilized Cyropreserved Peripheral Blood Stem Cell Units Depends on Monocyte Depletion. Biol Blood Marrow Transplant 2019; 25 (2): 223-32 doi 10.1016/j.bbmt.2018.10.004.
26. Stroncek D F, Ren J, Lee D W, Tran M, Frodigh S E, Sabatino M, et al. Myeloid cells in peripheral blood mononuclear cell concentrates inhibit the expansion of chimeric antigen receptor T cells. Cytotherapy 2016; 18 (7): 893-901 doi 10.1016/j.jcyt.2016.04.003.
27. Gauthier J, Gazeau N, Hirayama A V, Hill J A, Wu V, Cearley A, et al. Impact of CD19 CAR T-cell product type on outcomes in relapsed or refractory aggressive B-NHL. Blood 2022; 139 (26): 3722-31 doi 10.1182/blood.2021014497.
28. Melenhorst J J, Chen G M, Wang M, Porter D L, Chen C, Collins M A, et al. Decade-long leukaemia remissions with persistence of CD4 (+) CAR T cells. Nature 2022; 602 (7897): 503-9 doi 10.1038/s41586-021-04390-6.
29. Brentjens R, Yeh R, Bernal Y, Riviere I, Sadelain M. Treatment of chronic lymphocytic leukemia with genetically targeted autologous T cells: case report of an unforeseen adverse event in a phase I clinical trial. Mol Ther 2010; 18 (4): 666-8 doi 10.1038/mt.2010.31.
30. Locke F L, Neelapu S S, Bartlett N L, Siddiqi T, Chavez J C, Hosing C M, et al. Phase 1 Results of ZUMA-1: A Multicenter Study of KTE-C19 Anti-CD19 CAR T Cell Therapy in Refractory Aggressive Lymphoma. Mol Ther 2017; 25 (1): 285-95 doi 10.1016/j.ymthe.2016.10.020.
31. Kochenderfer J N, Dudley M E, Feldman S A, Wilson W H, Spaner D E, Maric I, et al. B-cell depletion and remissions of malignancy along with cytokine-associated toxicity in a clinical trial of anti-CD19 chimeric-antigen-receptor-transduced T cells. Blood 2012; 119 (12): 2709-20 doi 10.1182/blood-2011-10-384388.
32. Tong C, Zhang Y, Liu Y, Ji X, Zhang W, Guo Y, et al. Optimized tandem CD19/CD20 CAR-engineered T cells in refractory/relapsed B-cell lymphoma. Blood 2020; 136 (14): 1632-44 doi 10.1182/blood.2020005278.
33 Plaks V, Rossi J M, Chou J, Wang L, Poddar S, Han G, et al. CD19 target evasion as a mechanism of relapse in large B-cell lymphoma treated with axicabtagene ciloleucel. Blood 2021 doi 10.1182/blood.2021010930.
34. Majzner R G, Mackall C L. Tumor Antigen Escape from CAR T-cell Therapy. Cancer Discov 2018 doi 10.1158/2159-8290.CD-18-0442.
35. Zhang Y, Wang Y, Liu Y, Tong C, Wang C, Guo Y, et al. Long-term activity of tandem CD19/CD20 CAR therapy in refractory/relapsed B-cell lymphoma: a single-arm, phase 1-2 trial. Leukemia 2022; 36 (1): 189-96 doi 10.1038/s41375-021-01345-8.
36. Locke F L, Ghobadi A, Jacobson C A, Miklos D B, Lekakis L J, Oluwole O O, et al. Long-term safety and activity of axicabtagene ciloleucel in refractory large B-cell lymphoma (ZUMA-1): a single-arm, multicentre, phase 1-2 trial. Lancet Oncol 2019; 20 (1): 31-42 doi 10.1016/S1470-2045 (18) 30864-7.
37 Jacobson C A, Chavez J C, Sehgal A R, William B M, Munoz J, Salles G, et al. Axicabtagene ciloleucel in relapsed or refractory indolent non-Hodgkin lymphoma (ZUMA-5): a single-arm, multicentre, phase 2 trial. Lancet Oncol 2022; 23 (1): 91-103 doi 10.1016/S1470-2045 (21) 00591-X.
38 Fowler N H, Dickinson M, Dreyling M, Martinez-Lopez J, Kolstad A, Butler J, et al. Tisagenlecleucel in adult relapsed or refractory follicular lymphoma: the phase 2 ELARA trial. Nat Med 2022; 28 (2): 325-32 doi 10.1038/s41591-021-01622-0.
39 Schuster S J, Tam C S, Borchmann P, Worel N, McGuirk J P, Holte H, et al. Long-term clinical outcomes of tisagenlecleucel in patients with relapsed or refractory aggressive B-cell lymphomas (JULIET): a multicentre, open-label, single-arm, phase 2 study. Lancet Oncol 2021; 22 (10): 1403-15 doi 10.1016/S1470-2045 (21) 00375-2.
40. Abramson J S, Palomba M L, Gordon L I, Lunning M A, Wang M, Arnason J, et al. Lisocabtagene maraleucel for patients with relapsed or refractory large B-cell lymphomas (TRANSCEND NHL 001): a multicentre seamless design study. Lancet 2020; 396 (10254): 839-52 doi 10.1016/S0140-6736 (20) 31366-0.
41. Majzner R G, Mackall C L. Clinical lessons learned from the first leg of the CAR T cell journey. Nat Med 2019; 25 (9): 1341-55 doi 10.1038/s41591-019-0564-6.
42. Yu H, Sotillo E, Harrington C, Wertheim G, Paessler M, Maude S L, et al. Repeated loss of target surface antigen after immunotherapy in primary mediastinal large B cell lymphoma. Am J Hematol 2017; 92 (1): E11-E3 doi 10.1002/ajh.24594.
43 Beider K, Besser M J, Schachter J, Grushchenko-Polaq A H, Voevoda V, Wolf I, et al. Upregulation of Senescent/Exhausted Phenotype of CAR T Cells and Induction of Both Treg and Myeloid Suppressive Cells Correlate with Reduced Response to CAR T Cell Therapy in Relapsed/Refractory B Cell Malignancies. Blood 2019; 134:3234.
44. Lee D W, Santomasso B D, Locke F L, Ghobadi A, Turtle C J, Brudno J N, et al. ASTCT Consensus Grading for Cytokine Release Syndrome and Neurologic Toxicity Associated with Immune Effector Cells. Biol Blood Marrow Transplant 2019; 25 (4): 625-38 doi 10.1016/j.bbmt.2018.12.758.
45. Neelapu S S, Tummala S, Kebriaei P, Wierda W, Gutierrez C, Locke F L, et al. Chimeric antigen receptor T-cell therapy-assessment and management of toxicities. Nat Rev Clin Oncol 2018; 15 (1): 47-62 doi 10.1038/nrclinonc.2017.148.
46. Cheson B D, Fisher R I, Barrington S F, Cavalli F, Schwartz L H, Zucca E, et al. Recommendations for initial evaluation, staging, and response assessment of Hodgkin and non-Hodgkin lymphoma: the Lugano classification. J Clin Oncol 2014; 32 (27): 3059-68 doi 10.1200/JCO.2013.54.8800.
47. Cheson B D, Pfistner B, Juweid M E, Gascoyne R D, Specht L, Horning S J, et al. Revised response criteria for malignant lymphoma. J Clin Oncol 2007; 25 (5): 579-86 doi 10.1200/JCO.2006.09.2403.

What is claimed is:

1. A method for manufacturing a CD19/CD20 bi-specific chimeric antigen receptor (CAR) T cell comprising the ordered steps of:
(a) providing a composition comprising a population of cells comprising T cells
(b) contacting the composition comprising the population of cells with one or more of a transactivating composition, IL-2, and/or IL-15;
(c) transducing the composition comprising the population of cells from (b) with a CD19/CD20 bi-specific CAR nucleic acid;
(d) removing the transactivating composition from the transduced cell composition of (c); and
wherein the method further comprises contacting the cell with protamine sulfate within 24 hours of the cell transduction.

2. A method for manufacturing a CD19/CD20 bi-specific chimeric antigen receptor (CAR) T cell comprising the ordered steps of:
(a) providing a composition comprising a population of cells comprising T cells
(b) contacting the composition comprising the population of cells with one or more of a transactivating composition, IL-2, and/or IL-15;
(c) transducing the composition comprising the population of cells from (b) with a CD19/CD20 bi-specific CAR nucleic acid;
(d) removing the transactivating composition from the transduced cell composition of (c); and
wherein the transactivating composition comprises MACS® GMP T cell TransAct™.

3. The method of claim 1 or 2, wherein the composition comprising the population of cells from (a), (b), (c) and/or (d) comprises cell culture medium, wherein the cell culture medium is serum-free.

4. The method of any one of claims 1-3, wherein the population of cells comprising T cells are not depleted for CD14+ or CD25+ cells.

5. The method of any one of claims 1-4, wherein the method comprises contacting the composition with a transactivating composition and wherein the transactivating composition comprises MACS® GMP T cell TransAct™.

6. The method of claim 5, wherein the transactivating composition is used at a titer of 1:25-1:40.

7. The method of claim 5 or 6, wherein the transactivating composition is diluted to 1:35.

8. The method of any one of claims 1-7, wherein the population of cells in (a) comprises cells that have been isolated from a patient by leukapheresis.

9. The method of any one of claims 1-8, wherein the cell transduction is performed 24-72 hours after contacting a composition comprising the cell population with one or more of a transactivating composition, IL-2, and/or IL-15.

10. The method of any one of claims 1-9, wherein the transactivating composition is removed 6-7 days after first contact of the cells with the transactivating composition.

11. The method of any one of claims 1-10, wherein the cells are transduced with a lentiviral-based virus comprising a nucleic acid encoding the CAR.

12. The method of claim 11, wherein the virus is packaged in HEK-293T cells.

13. The method of any one of claims 1-12, wherein the method further comprises contacting the cell with protamine sulfate within 24 hours of the cell transduction.

14. The method of claim 13, wherein the final concentration of protamine sulfate is 10-20 μg/mL.

15. The method of claim 13 or 14, wherein the cells are transduced in a composition comprising protamine sulfate, IL-2, IL-15, and a virus comprising a nucleic acid encoding the CAR.

16. The method of claim 15, wherein the nucleic acid comprises a lentiviral vector backbone.

17. The method of any one of claims 1-16, wherein the nucleic acid encodes for a CAR polypeptide having an anti-CD19/CD20 binding region, and wherein the anti-CD19/CD20 binding region comprises SEQ ID NO:3.

18. The method of any one of claims 1-17, wherein the nucleic acid encodes for a CAR polypeptide comprising an IgG4 hinge region, CD28 transmembrane region, 4-1BB costimulatory region, and a CD3-zeta intracellular signaling domain.

19. The method of any one of claims 1-18, wherein the nucleic acid encodes for a CAR polypeptide having the sequence of SEQ ID NO:1 or a sequence with at least 80% sequence identity to SEQ ID NO:1.

20. The method of any one of claims 11-15, wherein the MOI of the virus is 0.1-1.5.

21. The method of any one of claims 1-20, wherein the cells are transduced at a concentration of 1×10⁶ cells/mL.

22. The method of any one of claims 1-21, wherein at least 1% cells are transduced.

23. The method of any one of claims 1-22, wherein at least 30% cells are viable at 3 days after transduction.

24. The method of any one of claims 11-21, wherein the cells are incubated with virus, protamine sulfate, IL-2 and IL-15 for 6-12 hours.

25. The method of any one of claims 1-24, wherein the cells are expanded 1.5-25 folds after transduction.

26. The method of any one of claims 1-25, wherein the method excludes enrichment or depletion after step (c).

27. The method of any one of claims 1-26, wherein the cell quantity is at least 1×10⁸-5×10⁹ cells by 14 days after step (a).

28. The method of any one of claims 1-27, wherein the method further comprises cryopreserving the cells.

29. The method of claim 28, wherein the cell quantity is at least 1×10⁸-5×10⁹ cells just prior to cryopreservation.

30. The method of claim 29, wherein the cells are cryopreserved at a concentration of 1×10⁶ cells/mL-15×10⁶ cells/mL

31. The method of any one of claims 28-28, wherein the cells are cryopreserved less than 17 days after transduction.

32. The method of any one of claims 28-31, wherein the cells are filtered prior to cryopreservation.

33. A population of CAR-T cells produced by the method of any one of claims 1-32.

34. The population of cells of claim 33, wherein the cells are thawed.

35. The population of cells of claim 33 or 34, wherein the population comprises at least 5% CD62L+CAR-T+ cells.

36. The population of cells of any one of claims 33-35, wherein the cells comprise at least 15% CD3+ cells.

37. The population of cells of any one of claims 33-36, wherein the cells have an average of 1-3 copies of the nucleic acid encoding the CAR per transduced cell.

38. The population of cells of any one of claims 34-37, wherein at least 70% of the cells are viable after thawing.

39. The population of cells of any one of claims 33-38, wherein the population comprises a mixture of CD4+ and CD8+ T cells.

40. The population of cells of claim 39, wherein at least 5% of the cells are CD4+.

41. The population of cells of any one of claims 39-40, wherein at least 15% of the cells are CD8+.

42. A composition comprising the population of cells according to any one of claims 33-41.

43. The composition of claim 42, wherein the composition comprises Isolyte®.

44. The composition of claim 42, wherein the composition comprises one or more of sodium chloride, sodium gluconate, sodium acetate, potassium chloride, magnesium chloride, sodium phosphate, dibasic, and potassium phosphate.

45. The composition of any one of claims 42-44, wherein the composition is buffered at pH 7.4.

46. A method for treating a subject for B-cell malignancy comprising administering the cells of any one of claims 33-41 or the composition of any one of claims 42-45.

47. The method of claim 46, wherein 1×10⁶-2×10⁸ cells are administered.

48. The method of claim 47, wherein the cells are determined to be positive for expression of the CAR.

49. The method of any one of claims 46-48, wherein the cells are autologous cells.

50. The method of any one of claims 46-49, wherein the B-cell malignancy is relapsed/refractory B-cell malignancy.

51. The method of any one of claims 46-50, wherein the subject has previously been treated for the B-cell malignancy.

52. The method of claim 51, wherein the subject has previously been treated with at least 1, 2 or at least 3 lines of therapy.

53. The method of claim 51 or 52, wherein the previous treatment comprises Bendamustine, Rituximab, Acalabrutinib, Umbralisib, Ublituximab, Lenalidomide, cyclophosphamide, doxorubicin, vincristine, prednisone, R-CHOP (combination of rituximab, cyclophosphamide, doxorubicin, vincristine, and prednisone), ifosfamide, carboplatin, etoposide, R-ICE (combination of rituximab, ifosfamide, carboplatin, and etoposide), R-EPOCH (combination of rituximab, etoposide phosphate, prednisone, vincristine sulfate, cyclophosphamide, and doxorubicin hydrochloride), ROR1-Targeting Antibody-Drug Conjugate, anti-CD19 bispecific T cell engager; gemcitabine, oxaliplatin, dexamethasone, autologous stem cell transplantation, and combinations thereof.

54. The method of any one of claims 46-50, wherein the subject has not previously been treated for the B-cell malignancy.

55. The method of any one of claims 46-54, wherein the B-cell malignancy comprises lymphoma or leukemia.

56. The method of claim 55, wherein the B-cell malignancy comprises non-Hodgkin B-cell lymphoma.

57. The method of claim 56, wherein the non-Hodgkin B-cell lymphoma is further classified as indolent non-Hodgkin lymphomas, follicular lymphoma, lymphoplasmacytic lymphoma, marginal zone lymphoma, nodal marginal zone lymphoma, gastric mucosa-associated lymphoid tissue (MALT) lymphoma, extragastric MALT lymphoma, mediterranean abdominal lymphoma, splenic marginal zone lymphoma, primary cutaneous anaplastic large cell lymphoma, diffuse large B-cell lymphoma, primary mediastinal B-cell lymphoma, follicular large cell lymphoma, anaplastic large cell lymphoma, cutaneous anaplastic large cell lymphoma, systemic anaplastic large cell lymphoma, extranodal NK-/T-cell lymphoma, lymphomatoid granulomatosis, angioimmunoblastic T-cell lymphoma, peripheral T-cell lymphoma, hepatosplenic T-cell lymphoma, subcutaneous panniculitis-like T-cell lymphoma, enteropathy-type intestinal T-cell lymphoma, intravascular large B-cell lymphoma, Burkitt lymphoma, lymphoblastic lymphoma, adult T-cell leukemia/lymphoma, mantle cell lymphoma, posttransplantation lymphoproliferative disorder, true histiocytic lymphoma, primary effusion lymphoma, or plasmablastic lymphoma.

58. The method of claim 55, wherein the B-cell malignancy comprises leukemia, and wherein the leukemia is further classified as chronic lymphocytic leukemia, small-lymphocytic leukemia, acute lymphocytic leukemia, acute myeloid leukemia, chronic myeloid leukemia, chronic myelomonocytic leukemia, pediatric leukemia, precursor B acute lymphoblastic leukemia, precursor T acute lymphoblastic leukemia, Burkitt's leukemia, acute biphenotypic leukemia, B-cell prolymphocytic leukemia, acute promyelocytic leukemia, acute myeloblastic leukemia, acute megakaryoblastic leukemia, hairy cell leukemia, T-cell prolymphocytic leukemia, large granular lymphocytic leukemia, adult T-cell leukemia, or clonal eosinophilias.

59. The method of any one of claims 46-58, wherein the subject is being treated with an additional therapy.

60. The method of any one of claims 46-59, wherein the method further comprises administration of an additional therapy.

61. The method of claim 60, wherein the additional therapy comprises a chemotherapy.

62. The method of claim 60 or 61, wherein the additional therapy comprises lymphodepletion.

63. The method of any one of claims 60-62, wherein the additional therapy comprises fludarabine and/or cyclophosphamide.

64. The method of any one of claims 60-63, wherein the additional therapy is given prior to administration of the cells.

65. The method of claim 64, wherein the additional therapy is given to the subject starting at five days prior to administration of the cells.

66. The method of any one of claims 63-65, wherein the subject is administered 30 mg/m²/day for 30 min of fludarabine and 500 mg/m²/day for 60 min of cyclophosphamide for 3 days.
